(19) United States
(12) Patent Application Publication
(10) Pub. No.: US 2022/0387461 A1
(43) Pub. Date: Dec. 8, 2022

(54) CANCER VACCINE

(71) Applicant: VICTORIA LINK LTD, Wellington (NZ)

(72) Inventors: Ian Francis HERMANS, Aro Valley (NZ); Gavin Frank PAINTER, Lower Hutt (NZ); Kef Kawin PRASIT, Wellington (NZ)

(21) Appl. No.: 17/640,395

(22) PCT Filed: Sep. 3, 2020
(86) PCT No.: PCT/NZ2020/050099

§ 371 Date: Mar. 4, 2022

(30) Foreign Application Priority Data
Sep. 4, 2019 (AU) 2019903260

(51) Int. Cl. A61K 31/7004; A61K 39/00; A61K 45/06; A61K 47/54; A61P 35/00

(52) U.S. Cl. CPC A61K 31/7004; A61K 39/0011; A61K 45/06; A61K 47/549; A61P 35/00

(57) ABSTRACT

The invention relates to a combination of a TLR-9 agonist and a conjugate of Formula (I) or pharmaceutically acceptable salt thereof. (Formula (I))

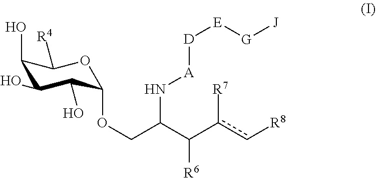

### 1. FIELD OF THE INVENTION

The invention relates generally to novel combinations of Toll-Like Receptor (TLR) agonists and glycolipid-peptide conjugates. These combinations can be used to treat cancerous tumours.

### 2. BACKGROUND

Although traditional cancer treatments (i.e. chemotherapy, radiation and surgery) can prolong lifespan for a number of cancer indications, they suffer from limitations in late stage disease. These limitations include inaccessibility for surgical resection and toxicity for both chemotherapeutic drugs and radiation treatment. The possibility of drug resistance and subsequent relapse is also limiting. The advent of clinically successful immunotherapies that unlock the patient's immune system to kill cancer cells, such as checkpoint blockade and infusion of T cells expressing tumour-specific chimeric antigen receptors (CARs), has dramatically changed the outlook for some cancer patients. For example, approximately 20-30% of metastatic melanoma patients respond to checkpoint blockade therapy. Response rates to CAR-T cell therapies can be higher, but these successes are limited to blood cancers without solid masses. Despite these successes, it is clear that the majority of cancer patients still do not respond to immunotherapy, which is particularly true for patients with solid tumours. For patients that do respond, current treatments can be associated with significant toxicity.

The main cause of cancer morbidity and mortality is metastasis, where cancer cells spread from the primary tumour to establish solid tumours in surrounding tissues and distant organs. Conventional therapies often become impractical in these patients. While there are suggestions that immunotherapy could be useful in this setting, there are significant barriers to ultimate clinical success of immunotherapy of solid tumours.

Perhaps the most significant barrier is the nature of the tumours themselves, be they primary or metastatic, with the solid masses typically developing a highly immunosuppressive microenvironment through release of tumour-derived factors, and infiltration of immunosuppressive leukocytes.

Accordingly, there is a great need in the art for cancer treatments, particularly treatments that are effective against unresectable and/or metastatic cancerous tumours.

It is therefore an object of the invention to provide combinations of TLR agonists and glycolipid-peptide conjugates that are useful for treating cancerous tumours and/or a method of treating cancerous tumours cancers using at least one combination of a TLR agonist and a glycolipid-peptide conjugate and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### 3. SUMMARY OF THE INVENTION

Disclosed herein is the inventor's work identifying combinations of TLR-9 agonists and glycolipid-peptide conjugates, and the use of such combinations in the treatment of cancerous tumours.

In one aspect the invention relates to a combination of a TLR-9 agonist and a conjugate of Formula (I) or pharmaceutically acceptable salt thereof;

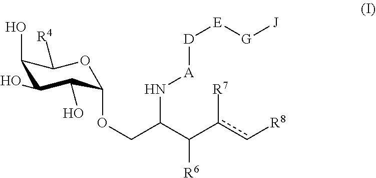

wherein:

A is a self-immolative linker group;

D is selected from the group consisting of:

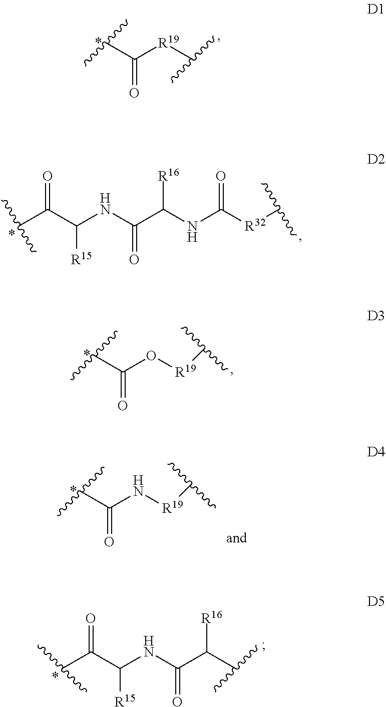

wherein * denotes a point of attachment of group D to group A;

R¹⁵ is a side chain of one of the following amino acids: L-lysine, L-citrulline, L-arginine, L-glutamine or L-threonine;

R¹⁶ is a side chain of a hydrophobic amino acid;

R¹⁹ is an alkylene group;

R³² is an alkylene group or an O-alkylene group wherein the O is attached to the carbonyl group of D2;

E is selected from the group consisting of:

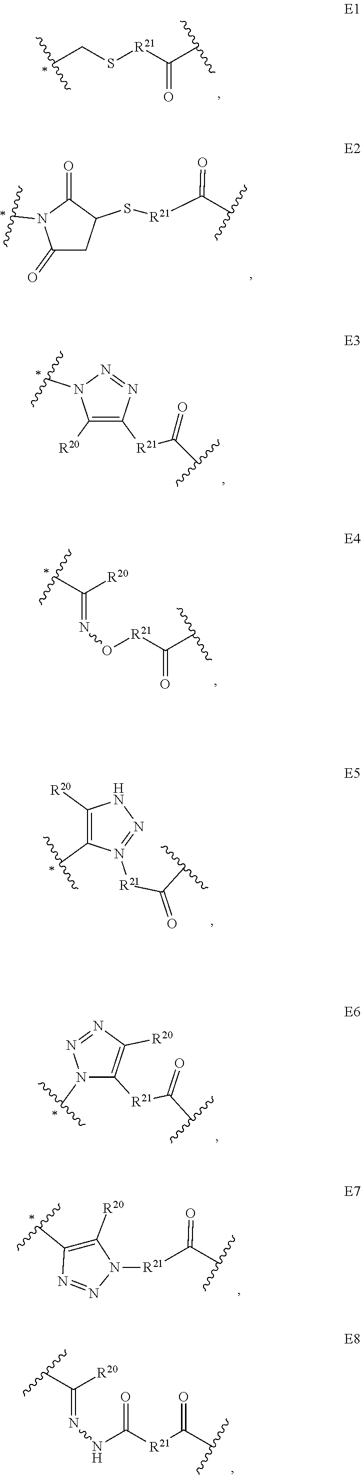

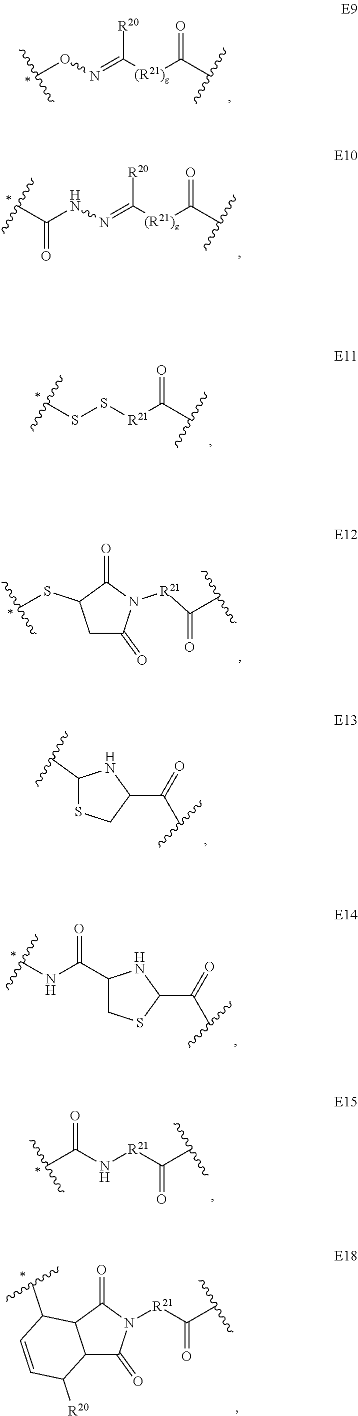

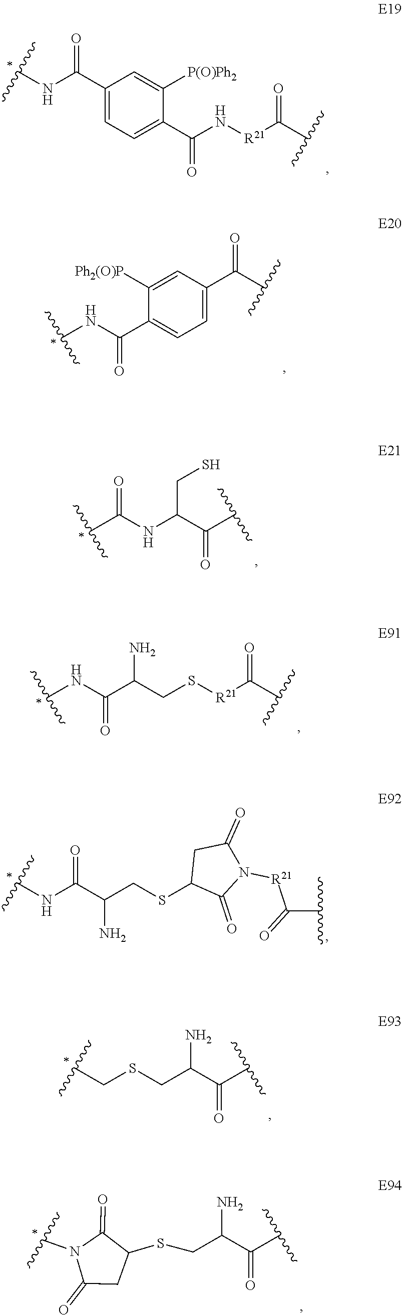

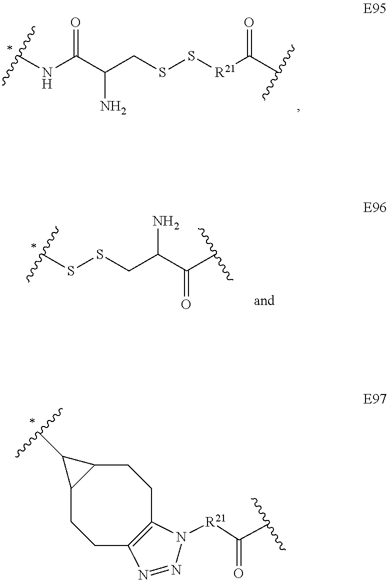

wherein * denotes a point of attachment of group E to group D;

R²⁰ is H or lower alkyl;

R²¹ is an alkylene group;

g is 0 when R²⁰ is H or g is 1 when R²⁰ is lower alkyl;

provided that E is E18 only when D is D1, D2 or D3 and provided that E is E1, E2, E3, E4, E5, E6, E7, E8, E9, E10, E11, E12, E13, E15, E20, E21, E93, E94 or E96 only when D is D1, D2, D3 or D4; and provided that E is E91, E92 or E95 only when D is D5 and provided that E is E97 only when D is D2;

G is absent or G is an amino acid sequence of up to 6 amino acids, attached through its N-terminus to group E and through its C-terminus to group J;

J is a peptide antigen, optionally substituted at its N and/or C-termini with up to 6 amino acids selected from the group of natural flanking residues for the antigen, and optionally terminated with NH₂ at the C-terminus so as to provide a C-terminal amide, and attached to group G through its N-terminus or, wherein G is absent, attached to group E through its N-terminus;

R⁴ is CH₃, CH₂OH, CH₂OCOR¹¹, CH₂OR¹¹, CH₂OSO₃H, CH₂SH, CH₂SR¹¹, CH₂SOR¹¹, CH₂SO₂R¹¹, CH₂PO₃H₂, CH₂OP(O)(OH)₂, CH₂OP(O)(OH)(OR¹¹), CH₂OP(O)(OR¹¹)₂, CO₂H, CH₂NHCOR¹¹, CH₂NHCO₂R¹¹, CH₂NHCONH₂, CH₂NHCONHR¹¹, CH₂NHCON(R¹¹)₂, CH₂N(R¹¹)₂, CH₂NHSO₂R¹¹;

R⁶ is OR¹², OH or H;

R⁷ is OR¹², OH or H; provided that at least one of R⁶ and R⁷ is OR¹²; wherein when R⁶ is OR¹², R⁷ is H, R⁸ is C₁-C₁₅ alkyl;

[symbol] denotes an optional double bond linking the carbon adjacent to R⁷ with the carbon adjacent to R⁸;

R⁸ is H or C₁-C₁₅ alkyl having a straight or branched carbon chain, wherein the carbon chain optionally incorporates one or more double bonds, one or more triple bonds, one or more oxygen atoms and/or a terminal or non-terminal optionally substituted aryl group;

R¹¹ is lower alkyl, lower alkenyl or aralkyl;

R¹² is C₆-C₃₀ acyl having a straight or branched carbon chain optionally substituted with one or more hydroxy groups at positions 2 and/or 3 of the acyl group and/or an optionally substituted chain terminating aryl group and which optionally incorporates one or more double bonds, one or more triple bonds, and/or one or more optionally substituted arylene groups and wherein the carbon chain is optionally substituted with one or more deuterium atoms; wherein the optional substituents on the aryl and arylene groups may be selected from halogen, cyano, dialkylamino, C₁-C₆ amide, nitro, C₁-C₆ alkoxy, C₁-C₆ acyloxy and C₁-C₆ thioalkyl.

In one aspect the invention relates to a combination of a TLR-9 agonist and a conjugate of Formula (II) or pharmaceutically acceptable salt thereof;

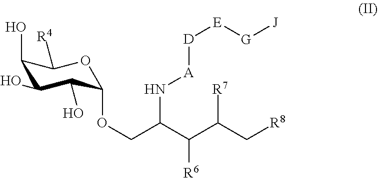

wherein R⁴, R⁶, R⁷, R⁸, A, D, E, G and J are all as defined above for Formula (I).

In one aspect the invention relates to a combination of a TLR-9 agonist and a conjugate of Formula (III) or pharmaceutically acceptable salt thereof;

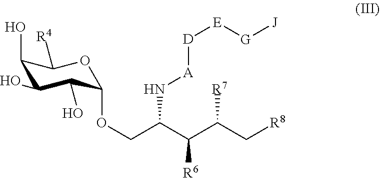

wherein R⁴, R⁶, R⁷, R⁸, A, D, E, G and J are all as defined above for formula (I).

In one aspect the invention relates to a combination of a TLR-9 agonist and a conjugate of Formula (IV) or pharmaceutically acceptable salt thereof;

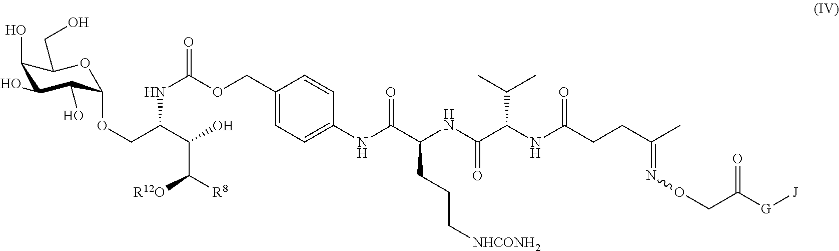

wherein R¹², R⁸, G and J are all as defined above for Formula (I).

In one aspect the invention relates to a combination of a TLR-9 agonist and a conjugate of Formula (V) or pharmaceutically acceptable salt thereof;

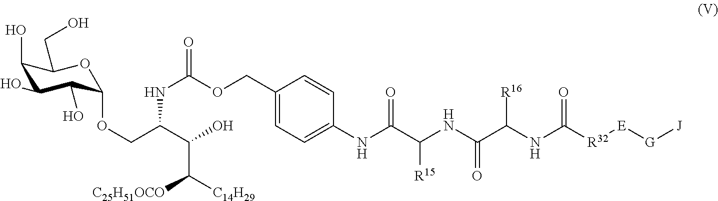

wherein R¹⁵, R¹⁶, E, G and J are all as defined above for Formula (I).

In one aspect the invention relates to a pharmaceutical composition comprising a combination of a TLR-9 agonist and a conjugate of any one of Formulae (I), (II), (III), (IV) or (V) or a pharmaceutically acceptable salt thereof, and a pharmaceutically acceptable carrier or excipient.

In one aspect the invention relates to a vaccine comprising a combination of a TLR-9 agonist and a conjugate of any one of Formulae (I), (II), (III), (IV) or (V) or a pharmaceutically acceptable salt thereof, and a pharmaceutically acceptable carrier or excipient.

In one aspect the invention relates to a kit comprising at least two unit dosage forms (A) and (B), wherein (A) comprises a TLR-9 agonist and (B) comprises a conjugate of any one of Formulae (I), (II), (III), (IV) or (V) or a pharmaceutically acceptable salt thereof;

and optionally (C) instructions for the simultaneous, sequential or separate administration of (A) and (B).

In one aspect the invention provides a method of inducing regression of a cancerous tumour in a subject, comprising administering to the subject a therapeutically effective amount of a combination of a TLR-9 agonist and a conjugate of any one of Formulae (I), (II), (III), (IV) or (V) or a pharmaceutically acceptable salt thereof, wherein the combination is administered by intratumoural or peritumoural injection, preferably intratumoural injection.

In another aspect the invention relates to a method of treating cancer in a subject, the method comprising administering to the subject a therapeutically effective amount of a combination of a TLR-9 agonist and a conjugate of any one of Formulae (I), (II), (III), (IV) or (V), or a pharmaceutically acceptable salt thereof; wherein the combination is administered by intratumoural or peritumoural injection to a cancerous tumour.

In another aspect the invention relates to a method of reducing the incidence of recurrence of cancer in a subject, the method comprising administering to the subject a therapeutically effective amount of a combination of a TLR-9 agonist and a conjugate of any one of Formulae (I), (II), (III), (IV) or (V), or a pharmaceutically acceptable salt thereof; wherein the combination is administered by intratumoural or peritumoural injection to a cancerous tumour.

Various embodiments of the different aspects of the invention as discussed above are also set out below in the detailed description of the invention, but the invention is not limited thereto. Other aspects of the invention may become apparent from the following description which is given by way of example only and with reference to the accompanying drawings.

### 4. BRIEF DESCRIPTION OF THE FIGURES

The invention will now be described by way of example only and with reference to the drawings in which:

FIG. 1 shows that intratumoural injection of CpG with a glycolipid-peptide conjugate is synergistic when compared to either agent on its own, providing a strong anti-tumour response with abscopal effect.

As described in Example 5, C57BL/6 mice were subcutaneously engrafted on both flanks with EG7.OVA tumours, a T lymphoma expressing ovalbumin (OVA) as a model of a tumour-associated antigen. Groups of these mice (n=8 per group) were then treated intratumourally on only one flank only with either vehicle only, CpG 1826 only, a conjugate comprising peptide epitopes from OVA linked to an iNKT cell agonist (CI-191), or combinations of CpG 1826 and conjugate CI-191, where the conjugate was either delivered at the same time as the first CpG or preceded the first CpG by 6 h. Tumours were allowed to become established (˜36 mm²) before treatment was initiated. The dosing regimen for CpG 1826 consisted of three treatments four days apart, where 3.9 nmol was injected directly into the tumour mass on two consecutive days (i.e., days 6 and 7, 10 and 11, 14 and 15). Treatment with conjugate was just once intratumourally with 2.3 nmol on day 6. Vehicle was administered with the same regimen as the delayed combination group. (A) Growth curves of injected and contralateral tumours in mice in each treatment group (B) Graph showing percent survival for each treatment group, where animals were culled when they reached ethical endpoint (>200 mm²).

FIG. 2 shows that intratumoural injection of CpG with a glycolipid-peptide conjugate induces a strong anti-tumour immune response (including abscopal effect).

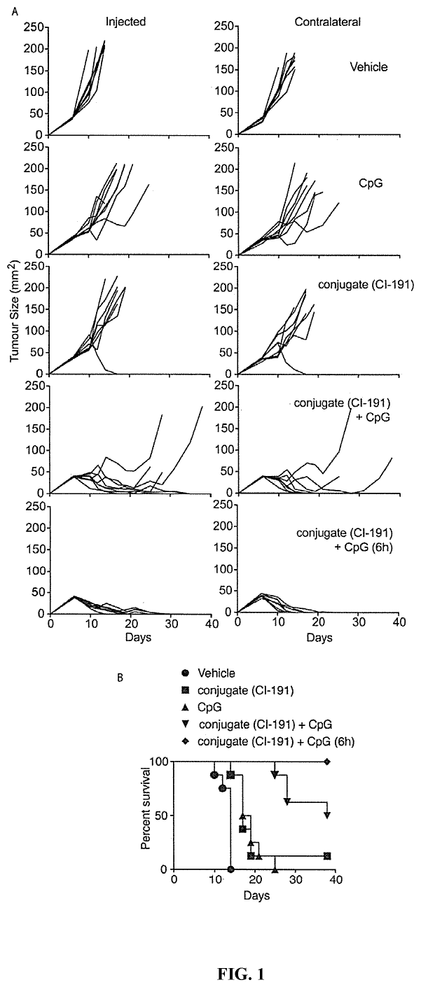

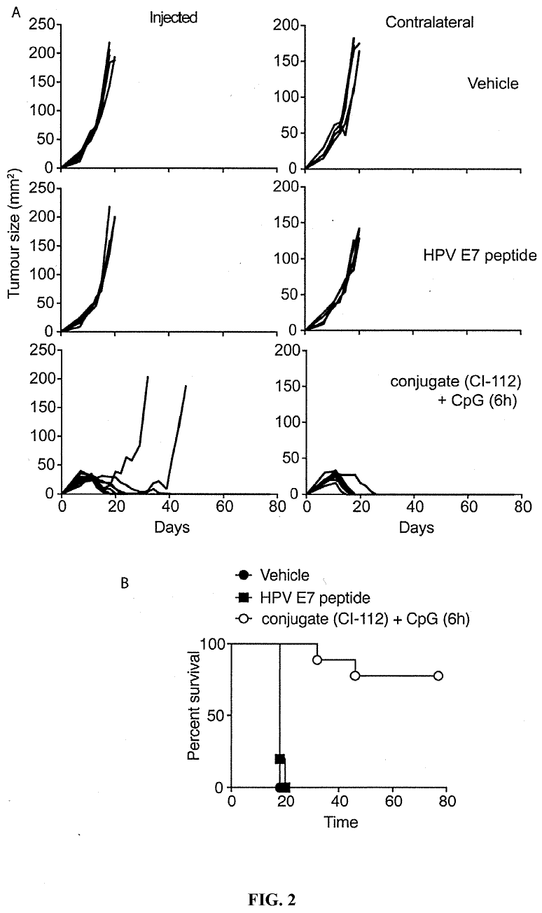

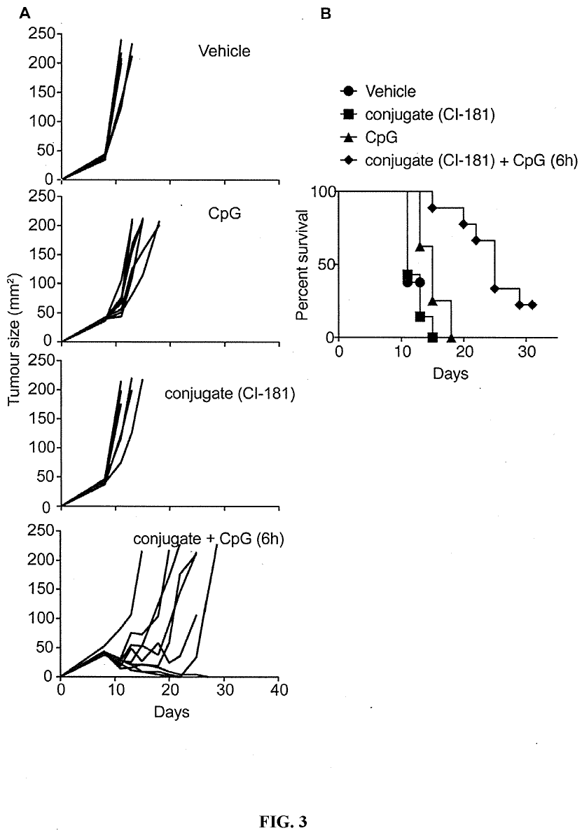

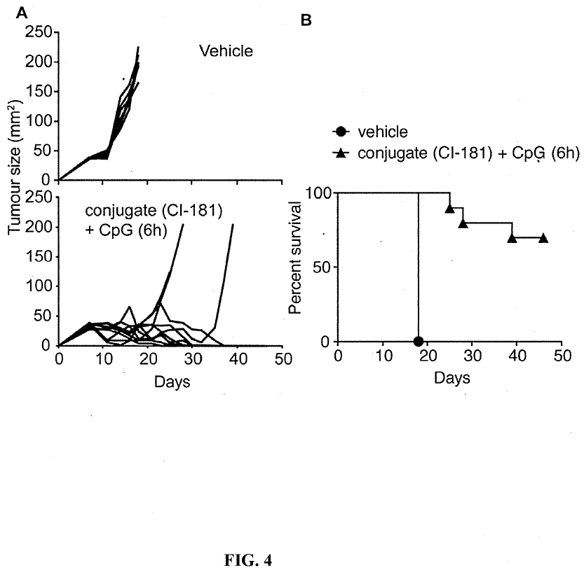

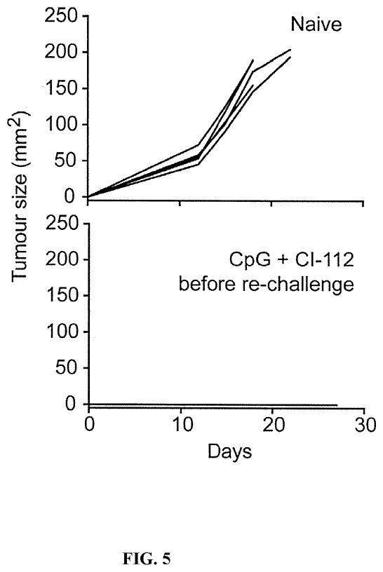

As described in Example 6, C57BL/6 mice were subcutaneously engrafted on both flanks with TC-1 tumours, a lung carcinoma expressing human papilloma virus (HPV) oncoproteins as a model of HPV-associated cancer. Groups of these mice (n=8 per group) were then treated intratumourally on only one flank with either vehicle only, 2.3 nmol of HPV E7 peptide (RAHYNIVTF) from HPV E7 oncoprotein or 2.3 nmol of conjugate comprising E7 epitope with flanking sequences linked to an iNKT cell agonist (CI-112) with repeated doses of 3.9 nmol of CpG 1826. Schedules for treatment with vehicle, CpG 1826 and conjugate were as in FIG. 1. Treatment with peptide was once, on day 6. (A) Growth curves of injected and contralateral tumours in mice in each treatment group (B) Graph showing percent survival for each treatment group, where animals were culled when they reached ethical endpoint (>200 mm²).

FIG. 3 shows that intratumoural injection of CpG with a mixture of glycolipid-peptide conjugates induces stronger antitumour immune responses than either agent alone

As described in Example 7, C57BL/6 mice were subcutaneously engrafted on one flank with B16.F10 tumours, a murine melanoma cell line known to express a range of neoantigens. Groups of these mice (n=8 per group) were then treated intratumourally with either vehicle only, repeated doses of CpG 1826 alone, or with one dose of a combination of 21 conjugates encompassing different neoepitopes linked separately to an iNKT cell agonist (CI-181) with repeated doses of CpG 1826. Schedules and doses for treatment with vehicle, CpG 1826 and conjugate (2.3 nmol) were as in FIG. 1. The conjugates CI-181 were given 6 h before the first dose of CpG 1826. (A) Growth curves of tumours in mice in each treatment group (B) Graph showing percent survival for each treatment group, where animals were culled when they reached ethical endpoint (>200 mm²).

FIG. 4 shows that intratumoural injection of CpG with a mixture of glycolipid-peptide conjugates induces a strong anti-tumour immune response

As described in Example 7, C57BL/6 mice were subcutaneously engrafted on one flank with B16.OVA tumours, a more immunogenic variant of B16.F10. Groups of these mice (n=10-11 per group) were then treated intratumourally with either vehicle only or with one dose of a combination of 21 conjugates encompassing different neoepitopes linked separately to an iNKT cell agonist (CI-181) with repeated doses of CpG 1826. Schedules for treatment with vehicle were as in FIG. 1. (A) Growth curves of tumours in mice in each treatment group (B) Graph showing percent survival for each treatment group, where animals were culled when they reached ethical endpoint (>200 mm²).

FIG. 5 shows that intratumoural injection of CpG with glycolipid-peptide conjugate induces anti-tumour immune responses that can prevent re-challenge with the same tumour

As described in Example 8 growth of TC-1 tumours was assessed in either naïve C57BL/6 mice (n=5), or C57BL/6 mice that had previously completely rejected TC-1 tumours after intratumoural treatment with glycolipid-peptide conjugate CI-112 and CpG 1826, where the first CpG administration was either at the time of the single CI-112 dose (n=6), or 6 h after CI-112 (n=7). The dosing regimen for this prior treatment was as in FIG. 1. Individual growth curves are shown, with all animals that had received prior therapy combined in the graph in the lower panel.

### 5. DETAILED DESCRIPTION OF THE INVENTION

### 5.1 Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains. It is also believed that practice of the present invention can be performed using standard microbiological, molecular biology, pharmacology and biochemistry protocols and procedures as known in the art.

The term “self-immolative linker” means any chemical group that, by covalent attachment, bridges a second and a third chemical group, wherein the covalent bond between the self-immolative linker and the second chemical group is metabolically cleavable in vivo and wherein, upon cleavage of this covalent bond in vivo, the self-immolative linker is detached from the second chemical group through spontaneous chemical bond rearrangements. At least one, preferably both, of the second and third chemical groups is a biologically active, e.g. pharmaceutically active, agent or prodrug thereof. In the glycolipid-peptide conjugates described herein, each of the second and third chemical groups is independently an immune stimulant (e.g. iNKT-cell agonist) or an antigen (e.g. peptide, protein or carbohydrate). In some examples, upon detachment of the self-immolative linker from the second chemical group, the self-immolative linker fragments and detaches from the third chemical group. Examples of self-immolative linkers are described in Philip L. Carl, Prasun K. Chakravarty, John A. Katzenellenbogen, Journal of Medicinal Chemistry, 1981, Vol. 24, No. 5, pg 479; and Simplicio et al., Molecules, 2008, vol. 13, pg 519. The covalent bond between the self-immolative linker and the second chemical group may be cleaved by, for example, an esterase, a peptidase, a phosphatase, a phospholipase or a hydrolase, or by way of a redox or pH-dependent process.

The term “alkyl” means any saturated hydrocarbon radical having up to 30 carbon atoms and includes any C1-C25, C1-C20, C1-C15, C1-C10, or C1-C6 alkyl group, and, unless specified otherwise, is intended to include cyclic (including fused bicyclic) alkyl groups (sometimes referred to herein as “cycloalkyl”), straight-chain and branched-chain alkyl groups, and straight or branched chain alkyl groups substituted with cyclic alkyl groups. Examples of alkyl groups include: methyl group (Me), ethyl group, n-propyl group, iso-propyl group, cyclopropyl group, n-butyl group, iso-butyl group, sec-butyl group, t-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group, cyclohexyl group, cyclooctyl group, and 1-methyl-2-ethylpropyl group.

In some embodiments, “alkyl” means any straight-chain saturated hydrocarbon radical having up to 30 carbon atoms.

The term “alkylene” means a diradical corresponding to an alkyl group. Examples of alkylene groups include methylene group, cyclohexylene group, ethylene group. An alkylene group can incorporate one or more cyclic alkylene group(s) in the alkylene chain, for example, “alkylene” can include a cyclohexylene group attached to a methylene group. Any alkylene group may be optionally substituted with one or more substituents selected from the group consisting of hydroxyl, halogen, e.g. fluorine, alkyl, e.g. methyl, and aryl. Any alkylene may optionally include one or more arylene moieties within the alkylene chain, for example, a phenylene group may be included within an alkylene chain.

The term “lower alkyl” means any saturated hydrocarbon radical having from 1 to 6 carbon atoms and is intended to include both straight- and branched-chain alkyl groups. Any alkyl group may optionally be substituted with one or more substituents selected from the group consisting of SO₃H (or a salt thereof), hydroxy and halogen, e.g. fluorine.

The term “alkenyl” means any hydrocarbon radical having at least one double bond, and having up to 30 carbon atoms, and includes any C2-C25, C2-C20, C2-C15, C2-C10 or C2-C6 alkenyl group, and is intended to include both straight- and branched-chain alkenyl groups. Examples of alkenyl groups include: ethenyl group, n-propenyl group, iso-propenyl group, n-butenyl group, iso-butenyl group, sec-butenyl group, t-butenyl group, n-pentenyl group, 1,1-dimethylpropenyl group, 1,2-dimethylpropenyl group, 2,2-dimethylpropenyl group, 1-ethylpropenyl group, 2-ethylpropenyl group, n-hexenyl group and 1-methyl-2-ethylpropenyl group.

The term “lower alkenyl” means any hydrocarbon radical having at least one double bond, and having from 2 to 6 carbon atoms, and is intended to include both straight- and branched-chain alkenyl groups.

Any alkenyl group may optionally be substituted with one or more substituents selected from the group consisting of alkoxy, hydroxy and halogen, e.g. fluorine.

The term “aryl” means an aromatic radical having 4 to 18 carbon atoms and includes heteroaromatic radicals. Examples include monocyclic groups, as well as fused groups such as bicyclic groups and tricyclic groups. Examples include a phenyl group (Ph), indenyl group, 1-naphthyl group, 2-naphthyl group, azulenyl group, heptalenyl group, biphenyl group, indacenyl group, acenaphthyl group, fluorenyl group, phenalenyl group, phenanthrenyl group, anthracenyl group, cyclopentacyclooctenyl group, and benzocyclooctenyl group, pyridyl group (Py), pyrrolyl group, pyridazinyl group, pyrimidinyl group, pyrazinyl group, triazolyl group (including a 1-H-1,2,3-triazol-1-yl and a 1-H-1,2,3-triazol-4-yl group), tetrazolyl group, benzotriazolyl group, pyrazolyl group, imidazolyl group, benzimidazolyl group, indolyl group, isoindolyl group, indolizinyl group, purinyl group, indazolyl group, furyl group, pyranyl group, benzofuryl group, isobenzofuryl group, thienyl group, thiazolyl group, isothiazolyl group, benzothiazolyl group, oxazolyl group, and isoxazolyl group.

The term “arylene” means a diradical corresponding to an aryl group. Examples include phenylene group.

The term “aralkyl” means an aryl group which is attached to an alkylene moiety, where aryl and alkylene are as defined above. Examples include benzyl group.

Any aryl or aralkyl group may optionally be substituted with one or more substituents selected from the group consisting of alkyl, halogen, cyano, dialkylamino, amide (both N-linked and C-linked: —NHC(O)R and —C(O)NHR), nitro, alkoxy, acyloxy and thioalkyl. The term “alkoxy” means an OR group, where R is alkyl as defined above.

The term “lower alkoxy” means an OR group, where R is “lower alkyl” as defined above. The term “acyl” means a C(═O)R′ group, where R′ is alkyl as defined above. The term “fatty-acyl group” is a group formed by loss of OH from the carboxy group of a fatty acid.

The term “acyloxy” means an OR″ group, where R″ is acyl as defined above.

The term “amino acid” includes both natural and non-natural amino acids. Amino acids may be referred to using the three letter code or one letter code, as understood by a person skilled in the art.

The term “amide” includes both N-linked (—NHC(O)R) and C-linked (—C(O)NHR) amides.

The term “pharmaceutically acceptable salt” is intended to apply to non-toxic salts derived from inorganic or organic acids, including, for example, the following acid salts: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, p-toluenesulfonate, salicylate, succinate, sulfate, tartrate, thiocyanate, and undecanoate.

The term “antigen” refers to a molecule that contains one or more epitopes (linear, overlapping, conformational or a combination of these) that, upon exposure to a subject, can induce an immune response in the subject, that is specific for that antigen.

The term “neoantigen” refers to a tumour antigen that arises from a tumour-specific mutation(s) which alters the amino acid sequence of genome-encoded proteins.

The term “epitope” refers to an antigenic determinant, i.e. a point of interaction on the antigen for specific antibodies or an antigenic determinant that is presented on an MHC molecule and recognized by a T-cell receptor. An antigen may contain more than one antigenic determinant.

The term “TLR-9 agonist” as used herein, refers to an agent that is an agonist for the Toll-like receptor 9 (TLR-9). TLR-9 is a member of the TLR family. It is a receptor expressed in immune system cells such as dendritic cells, macrophages, natural killer cells and other antigen presenting cells. TLR-9 binds DNA present in bacteria and viruses, triggering a signalling cascade that generates a pro-inflammatory cytokine response. Preferred TLR-9 agonists are CpG oligonucleotides.

The terms “CpG oligodeoxynucleotide” or “CpG” as used herein refer to a DNA molecule containing a cytoside base (C) followed by a guanine base (G). CpG oligonucleotides suitable for use in the combinations of the invention include Type A, in which the bases are linked by phosphodiester linkages; Type B, in which the bases are linked by phosphorothioate linkages, and Type C which comprise an oligonucleotide sequence and its 3′ palindrome which together form a duplex. Generally, a type A CpG oligdeoxynucleotide will incorporate poly G residues at the termini to increase stability and promote the formation of higher order structures. Each oligodeoxynucleotide will generally contain 2-4 CpG motifs which can be defined as the CpG plus the 2 bases either side. The CpG oligodeoxynucleotide GTCGTT is preferred for human applications. Generally individual CpG motifs are separated by at least 2 bases which are often thymine.

The terms “administering” or “administration” refer to placement of a combination of the invention or one of its components into a subject by a method appropriate to result in a cancer treatment.

The terms “cancer” and “cancerous tumour” as used herein, refer to cells that have undergone a malignant transformation or cellular changes that result in aberrant or unregulated growth or hyperproliferation. Such changes or malignant transformations usually make such cells pathological to the host organism. Precancers and precancerous cells that are or could become pathological and require or could benefit from intervention are also intended to be included. Cancerous tumours may be solid or hematopoietic. Hematopoietic tumours are liquid tumours of blood cells. Specific examples of clinical conditions based on hematopoietic tumours include leukemia such as chronic myelocytic leukemia or acute myelocytic leukemia, myeloma such as multiple myeloma; lymphoma and the like.

The term “intratumoural injection” as used herein with reference to administration of an agent, means the direct injection of the agent into a tumour.

The term “peritumoural injection”, as used herein with reference to administration of an agent, means the direct injection of the agent into the peritumoural region of a tumour.

The term “injected tumour” as used herein means a tumour which has received an intratumoural or peritumoural injection. In one embodiment, the injected tumour is at least about 30 mm² (as measured by the product of bisecting diameters).

The term “distal tumour” as used herein means a tumour that is not an injected tumour, and which is located at a different position in the subject from the injected tumour.

The term “primary tumour” as used herein refers to the original or first tumour in the body of a subject. Primary cancer cells (that is, cells obtained from near the site of malignant transformation) can be readily distinguished from non-cancerous cells by well-established techniques, particularly histological examination. Primary cancer cells from the primary tumour may spread to other parts of the body and form new metastatic tumours.

The term “abscopal effect” as used herein refers to the phenomenon of tumour regression at a site distant from the injected tumour.

The terms “reduce” and “decrease” (and grammatical variations thereof) as used herein with reference to the incidence of recurrence of a cancer, mean a measurable or observable reduction or decrease in the recurrence of the cancer in a subject treated with a combination of the invention relative to the recurrence of the cancer observed in an appropriate control (e.g., untreated) subject; e.g., placebo or non-active agent. In preferred embodiments the measurable or detectable decrease or reduction is a statistically significant increase, relative to an appropriate control.

The term “regression” (and grammatical variations thereof) as used herein with reference to treatment of a cancerous tumour means that the tumour reduces in volume relative to its volume prior to treatment. A treated tumour may regress by at least 5, 10, 15, 20, 25, 30, 35, 40, 45, 50, 55, 60, 65, 70, 75, 80, 85, 90, 95, 99% or 100%. 100% regression occurs when a cancerous tumour shrinks in volume so as to be clinically undetectable, for example, by procedures such as CAT scan, MR imagine, X-ray, ultrasound or palpation, or the expression of one or more cancer-specific antigens in a sample obtained from a patient.

The term “therapeutically effective amount” (or “effective amount”) refers to an amount sufficient to effect beneficial or desired results, including clinical results, but not limited thereto. A therapeutically effective amount of the combination of the invention can be administered in one or more administrations of each agent separately or together. The therapeutically effective amount of the agents to be administered to a subject depends on, for example, the purpose for which the agents are administered, mode of administration, nature and dosage of any co-administered compounds, and characteristics of the subject, such as general health, other diseases, age, sex, genotype, body weight and tolerance to drugs. A person skilled in the art will be able to determine appropriate dosages having regard to these any other relevant factors.

In the context of the present disclosure, a therapeutically effective amount of the combination of agents that is useful for treating cancer is the amount of the agents that is expected to be effective in a human based on the mouse data disclosed herein. Such an amount can be determined by the skilled worker using an appropriate conversion model.

The term “subject” refers to a human or a non-human animal, preferably a vertebrate that is a mammal. Non-human mammals include, but are not limited to; livestock, such as, cattle, sheep, swine, deer, and goats; sport and companion animals, such as, dogs, cats, and horses; and research animals, such as, mice, rats, rabbits, and guinea pigs. Preferably, the subject is a human.

The term “treating” and grammatical variations thereof as used herein refers to both therapeutic and prophylactic or preventative measures, wherein the object is to prevent or slow down the targeted conditions. For example, a subject is treated for cancer if, after receiving a therapeutic dose of agent, the subject shows an observable and/or measurable reduction in the number of cancer cells or absence of the cancer cells; reduction in tumour size, inhibition of tumour metastasis, inhibition of tumour growth, increase in length of remission and/or relief to some extent, of one or more symptoms associated with the cancer the subject is suffering from, including reduced morbidity and mortality and/or improvement in quality of life.

The term “vaccine” and grammatical variations as used herein refers to a substance that stimulates an immune response, i.e., that induces the activation of immune cells that provide immunity against disease and/or reduce disease.

The term “pharmaceutically acceptable carrier or excipient” means a excipient or carrier that is compatible with the other ingredients of the composition, and not harmful to the subject to whom the composition is administered.

For the purposes of the invention, any reference to the disclosed agents of the combination includes all possible formulations, configurations, and conformations, for example, in free form (e.g. as a free acid or base), in the form of salts or hydrates, in the form of isomers (e.g. cis/trans isomers), stereoisomers such as enantiomers, diastereomers and epimers, in the form of mixtures of enantiomers or diastereomers, in the form of racemates or racemic mixtures, or in the form of individual enantiomers or diastereomers. Specific forms of the agents are described in detail herein.

The term “about” when used in connection with a referenced numeric indication means the referenced numeric indication plus or minus up to 10% of that referenced numeric indication. For example, “about 100” means from 90 to 110 and “about six” means from 5.4 to 6.6.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### 5.2 The Combinations of the Invention

The invention relates to novel combinations of TLR-9 agonists and glycolipid-peptide conjugates of Formulae (I), (II), (III), (IV) and (V). These agents, used in combination, comprise vaccines for use in the treatment of cancerous tumours.

The main cause of cancer morbidity and mortality is metastasis, where cancer cells spread from the primary tumour to establish solid tumours in surrounding tissues and distant organs. Conventional therapies often become impractical in these patients. The clinical success of checkpoint blockade in metastatic melanoma suggests that immunotherapy can work in this setting, eliciting systemic immune responses that attack tumours at the different sites. However, melanoma is generally regarded as a highly mutated tumour, and part of this success may reflect expression of a broad range of mutated proteins that can serve as antigenic targets. Also, checkpoint blockade is generally more effective when tumours already contain immune infiltrates, suggesting that the tumours are immunogenic, and that some limited anti-tumour immunity has been induced naturally in the patient before treatment.

Immunization, or vaccination, involves the administration of a substance (an antigen) to a patient in order to induce an immune response against said antigen. The purpose of immunization can be to prevent a disease (prophylactic immunization) or to treat an existing disease (therapeutic immunization). Antigens can be derived from pathogens or can be disease-related, e.g. an antigen found in tumour cells but not in normal cells used for a therapeutic immunization against cancer.

Specific immune responses against antigens can often be further stimulated by the co-administration of adjuvants. Adjuvants are known in the art to accelerate, prolong, or enhance the quality of the specific immune response to antigens. Many different types of adjuvants have been described in the art.

One set of adjuvants act through toll-like receptors (TLRs). TLRs recognize specific patterns of microbial components, especially those from pathogens, and regulate the activation of both innate and adaptive immunity. Thirteen members of the TLR-family have been identified in man. TLRs are expressed by phagocytic cells such as monocytes, macrophages and dendritic cells. A known group of adjuvants which interact with Toll-Like Receptor 9 (TLR-9) are the unmethylated cytosine-phosphate-guanine (CpG) dinucleotides.

TLR-9 is expressed by numerous cells of the immune system, including B cells, monocytes, natural killer (NK) cells, keratinocytes, melanocytes, and both conventional and plasmacytoid dendritic cells. In general, TLR-9 signals lead to pro-inflammatory reactions that result in the production of cytokines such as type-I interferon, IL-6, TNF, IFNα, and IL-12. The adjuvant effect of TLR-9 agonists is mediated via direct and indirect activation of antigen-presenting cells, notably dendritic cells, that are engaged in stimulating cells of the adaptive immune system.

In this context, activation of plasmacytoid dendritic cells to produce large quantities of type I interferon may play a role, with type I interferon known to enhance T cell stimulation by dendritic cells, and to also have a direct effect on T cell differentiation into memory cells. TLR-9 agonists also induce activation of B cells, and enhance differentiation of B cells into antibody-secreting plasma cells. The key biological role for TLR-9 is to alert the immune system to viral or bacterial infection. TLR-9 is expressed intracellularly within the endosomal compartments, where it can come into contact with acquired material from infected tissue, and functions by binding to DNA that is rich in CpG motifs, which is characteristic of many infectious agents.

Numerous early clinical trials using TLR-9 agonists in monotherapies for solid tumours and haematological malignancies provided evidence of CpG tolerability and safety. However, these studies failed to demonstrate sufficient anti-tumour efficacy.

Another type of adjuvant activity is that mediated through invariant natural killer T (iNKT) cells. iNKT cells recognise, and can respond to, glycolipids that are presented on the cell surface by the antigen-presenting molecule CD1d. Once activated through recognition of CD1d/glycolipid complexes, iNKT cells can induce activation dendritic cells, the most potent antigen-presenting cells in the body. If those same dendritic cells have acquired, and are presenting, antigens that can be recognised by T cells, a strong antigen-specific T cell response is induced. The glycolipid alpha-galactosylceramide (α-GalCer) is an example of an iNKT cell agonist, which has been shown to function as a potent vaccine adjuvant when co-administered with disease-relevant antigens in the treatment and prophylaxis of cancer and infectious disease.

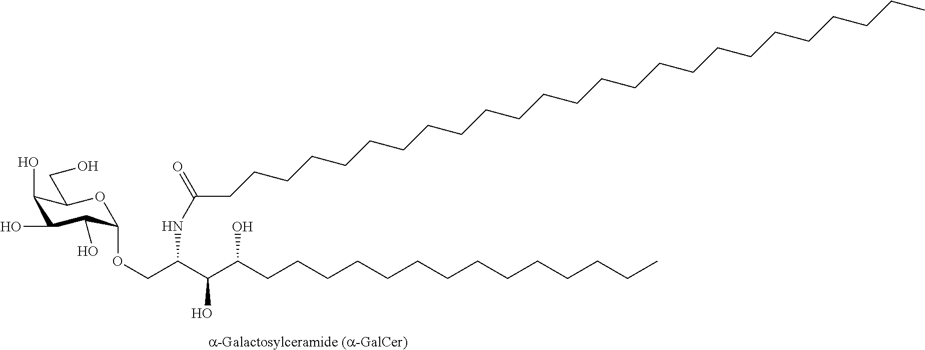

WO2014/088432 describes glycolipid-peptide conjugates in which an α-GalCer derivative is conjugated to a peptide antigen. The compound of Formula (I) below provides an example of such a glycolipid-peptide conjugate. When administered in vivo, conjugates of Formula (I) are enzymatically cleaved (at the A position of the conjugate) to release the peptide antigen component from the glycolipid molecule. Without being bound by theory, it is believed that O→N acyl migration between the 2 and 3-positions of the glycolipid molecule provides the corresponding amide, i.e. α-GalCer.

The inventors have now shown that the combination of a TLR-9 agonist and a glycolipid-peptide conjugate of Formula (I) induces regression of cancerous tumours, when administered intratumourally. Administration of the combination was also observed to generate an abscopal effect in which regression of the injected tumour was accompanied by regression of tumours other than the injected tumour. Consequently, the combination of the invention provides a useful treatment for cancerous tumours.

In one aspect the invention relates to a combination of a TLR-9 agonist and a conjugate of Formula (I) or pharmaceutically acceptable salt thereof;

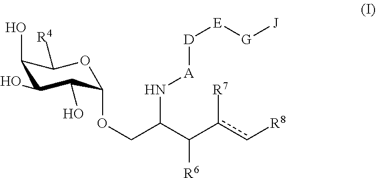

wherein:

A is a self-immolative linker group;

D is selected from the group consisting of:

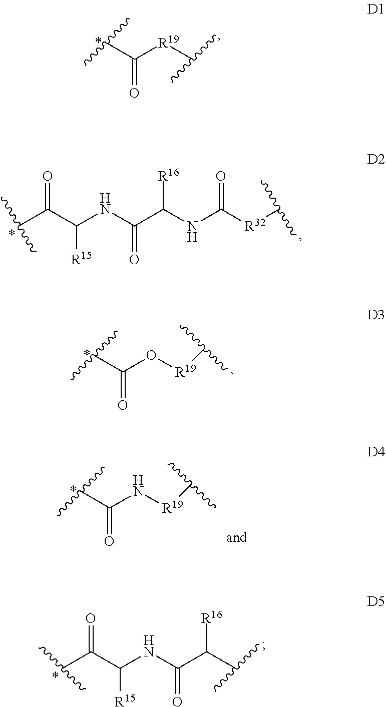

wherein * denotes a point of attachment of group D to group A;

R¹⁵ is a side chain of one of the following amino acids: L-lysine, L-citrulline, L-arginine, L-glutamine or L-threonine;

R¹⁶ is a side chain of a hydrophobic amino acid;

R¹⁹ is an alkylene group;

R³² is an alkylene group or an O-alkylene group wherein the O is attached to the carbonyl group of D2;

E is selected from the group consisting of:

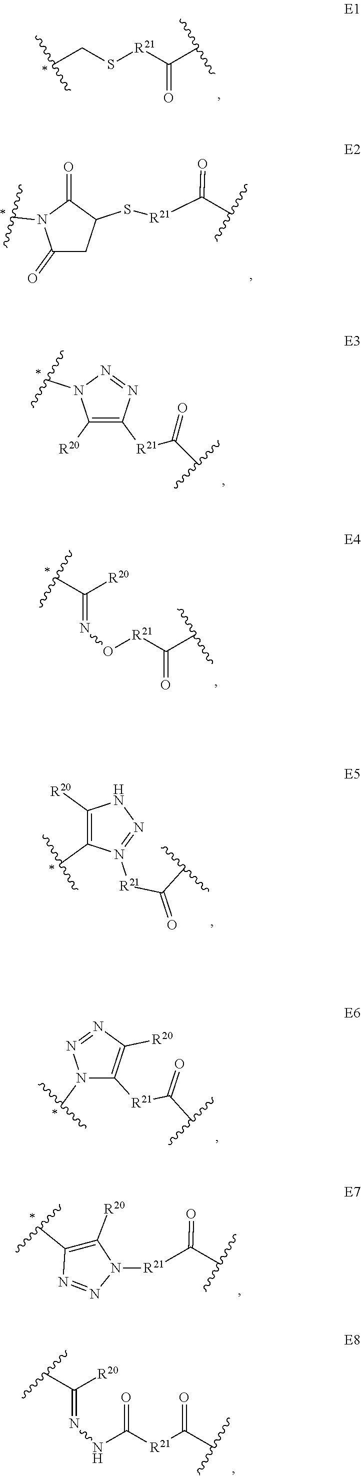

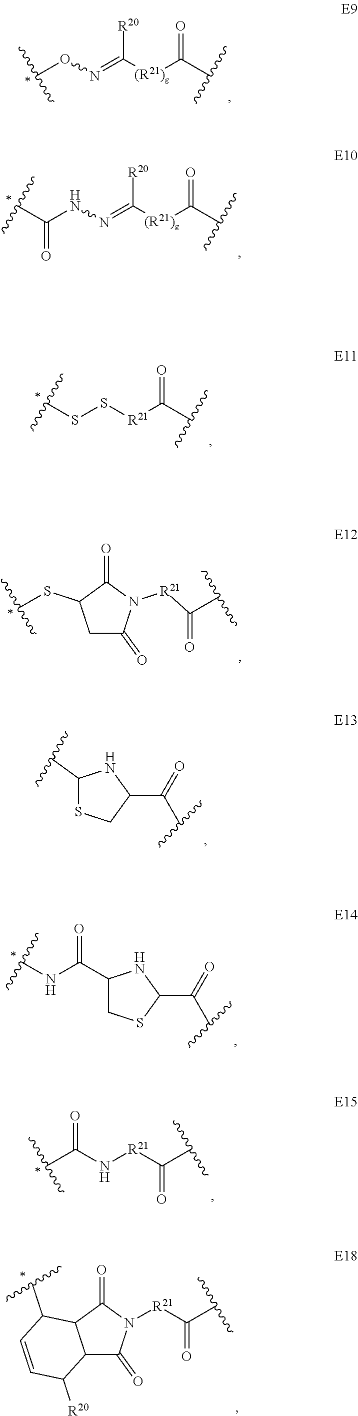

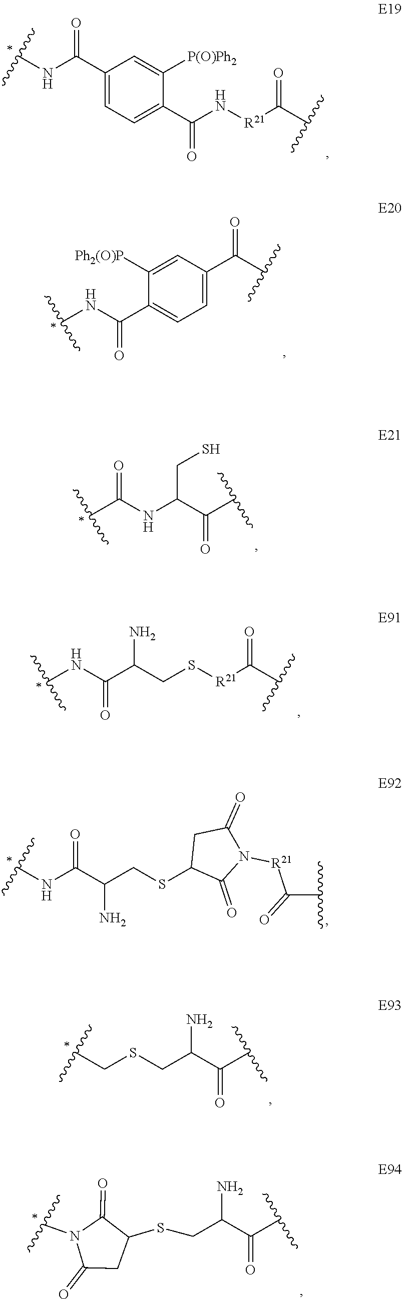

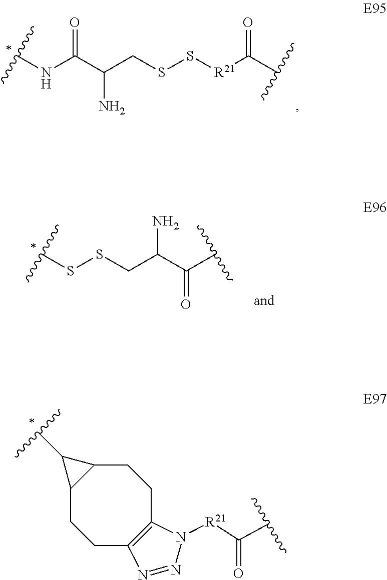

wherein * denotes a point of attachment of group E to group D;

R²⁰ is H or lower alkyl;

R²¹ is an alkylene group;

g is 0 when R²⁰ is H or g is 1 when R²⁰ is lower alkyl;

provided that E is E18 only when D is D1, D2 or D3 and provided that E is E1, E2, E3, E4, E5, E6, E7, E8, E9, E10, E11, E12, E13, E15, E20, E21, E93, E94 or E96 only when D is D1, D2, D3 or D4; and provided that E is E91, E92 or E95 only when D is D5 and provided that E is E97 only when D is D2;

G is absent or G is an amino acid sequence of up to 6 amino acids, attached through its N-terminus to group E and through its C-terminus to group J;

J is a peptide antigen, optionally substituted at its N and/or C-termini with up to 6 amino acids selected from the group of natural flanking residues for the antigen, and optionally terminated with NH₂ at the C-terminus so as to provide a C-terminal amide, and attached to group G through its N-terminus or, wherein G is absent, attached to group E through its N-terminus;

R⁴ is CH₃, CH₂OH, CH₂OCOR¹¹, CH₂O R¹¹, CH₂OSO₃H, CH₂SH, CH₂SR¹¹, CH₂SOR¹¹, CH₂SO₂R¹¹, CH₂PO₃H₂, CH₂OP(O)(OH)₂, CH₂OP(O)(OH)(OR¹¹), CH₂OP(O)(OR¹¹)₂, CO₂H, CH₂NHCOR¹¹, CH₂NHCO₂R¹¹, CH₂NHCONH₂, CH₂NHCONHR¹¹, CH₂NHCON(R¹¹)₂, CH₂N(R¹¹)₂, CH₂NHSO₂R¹¹;

R⁶ is OR¹², OH or H;

R⁷ is OR¹², OH or H; provided that at least one of R⁶ and R⁷ is OR¹²; wherein when R⁶ is

OR¹², R⁷ is H, R⁸ is C₁-C₁₅ alkyl;

[symbol] denotes an optional double bond linking the carbon adjacent to R⁷ with the carbon adjacent to R⁸;

R⁸ is H or C₁-C₁₅ alkyl having a straight or branched carbon chain, wherein the carbon chain optionally incorporates one or more double bonds, one or more triple bonds, one or more oxygen atoms and/or a terminal or non-terminal optionally substituted aryl group;

R¹¹ is lower alkyl, lower alkenyl or aralkyl;

R¹² is C₆-C₃₀ acyl having a straight or branched carbon chain optionally substituted with one or more hydroxy groups at positions 2 and/or 3 of the acyl group and/or an optionally substituted chain terminating aryl group and which optionally incorporates one or more double bonds, one or more triple bonds, and/or one or more optionally substituted arylene groups and wherein the carbon chain is optionally substituted with one or more deuterium atoms; wherein the optional substituents on the aryl and arylene groups may be selected from halogen, cyano, dialkylamino, C₁-C₆ amide, nitro, C₁-C₆ alkoxy, C₁-C₆ acyloxy and C₁-C₆ thioalkyl.

In one aspect the invention relates to a combination of a TLR-9 agonist and a conjugate of Formula (II) or pharmaceutically acceptable salt thereof;

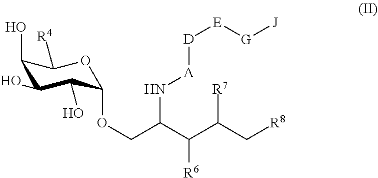

wherein R⁴, R⁶, R⁷, R⁸, A, D, E, G and J are all as defined above for Formula (I).

In one aspect the invention relates to a combination of a TLR-9 agonist and a conjugate of Formula (III) or pharmaceutically acceptable salt thereof;

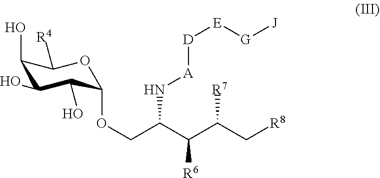

wherein R⁴, R⁶, R⁷, R⁸, A, D, E, G and J are all as defined above for Formula (I).

In one aspect the invention relates to a combination of a TLR-9 agonist and a conjugate of Formula (IV) or pharmaceutically acceptable salt thereof;

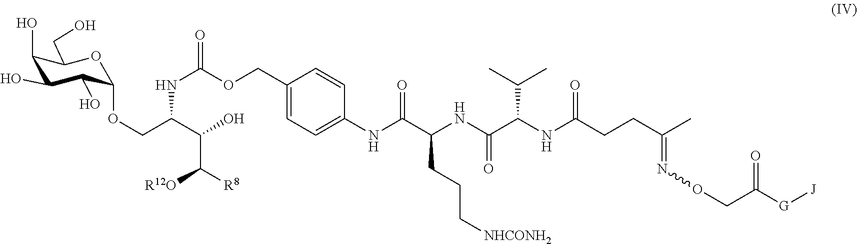

wherein R¹², R⁸ G and J are as defined above for Formula (I).

In one aspect the invention relates to a combination of a TLR-9 agonist and a conjugate of Formula (V) or pharmaceutically acceptable salt thereof;

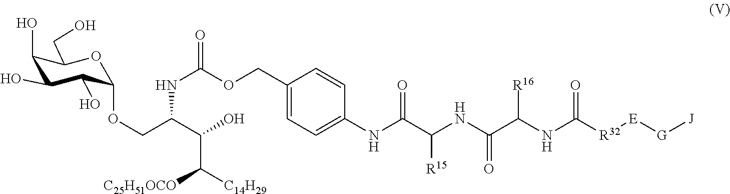

wherein R¹⁵, R¹⁶, E, G and J are as defined above for formula (I).

The glycolipid-peptide conjugates for use in the combination of the invention can be prepared in accordance with the methods outlined in WO2014/088432, incorporated herein by reference.

The following embodiments of the invention relate to the above conjugates of Formulae (I), (II), (III), (IV) and (V) as applicable:

In one embodiment, A is selected from the group consisting of:

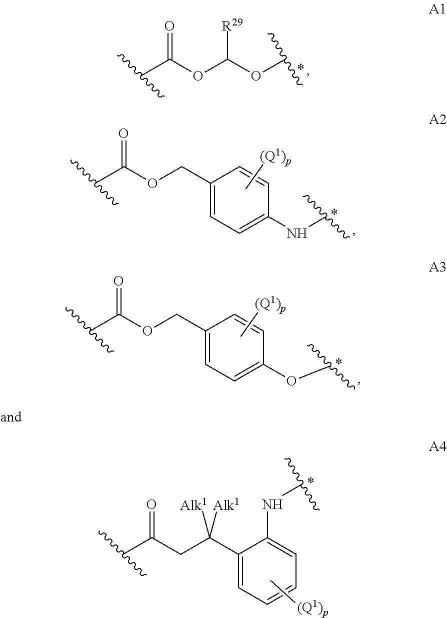

wherein * denotes a point of attachment of group A to group D;

each Q¹, the same or different, is independently selected from the group consisting of H, alkyl, alkoxy, halogen, nitro, aryl; or, together with the ring to which it is attached, forms a fused bicyclic aryl group;

p is an integer from 1 to 4;

Alk¹ is C₁-C₄ straight chain alkyl; and

R²⁹ is H or lower alkyl;

provided that A is A1 only when D is D1 and provided that A is A2 only when D is D2, D3 or D5 and provided that A is A3 only when D is D1, D3 or D4 and provided that A is A4 only when D is D2, D3 or D5.

In one embodiment A is selected from A2, A3 and A4 and Q¹ in A2, A3 and A4 is H.

In one embodiment, A is A2. In one embodiment, A is A2 wherein Q¹ is H.

In one embodiment, D is D2.

In one embodiment, R¹⁵ is the side chain of L-citrulline or L-alanine, preferably L-citrulline.

In one embodiment, R¹⁶ is a side chain of one of the following amino acids: L-phenylalanine, L-valine, L-leucine, L-isoleucine, L-norleucine, L-methionine, L-tryptophan or L-tyrosine; that is, preferably R¹⁶ is selected from the group consisting of:

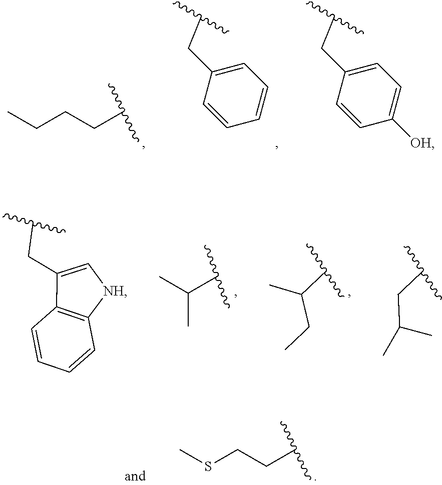

In one embodiment, R¹⁶ is selected from the group consisting of:

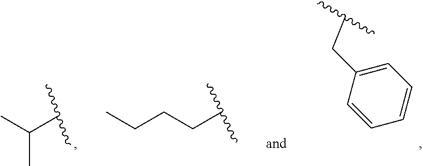

preferably

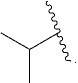 [SMILES: *C(C)C]

In one embodiment R³² is alkylene. In one embodiment, R³² is (C₆-C₈)-alkylene, preferably C₆-alkylene.

In one embodiment D is D2 wherein R¹⁵ is

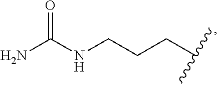 [SMILES: *CCCNC(N)=O]

### R¹⁶ is

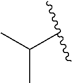 [SMILES: *C(C)C]

and R³² is C₆-alkylene.

In one embodiment, E is any one of E1 to E8, E93 or E94.

In one embodiment, E is any one of E1 to E4, E93, E94 or E97.

In one embodiment E is selected from the group consisting of E3, E4, E93, E94 and E97.

In one embodiment E is selected from the group consisting of:

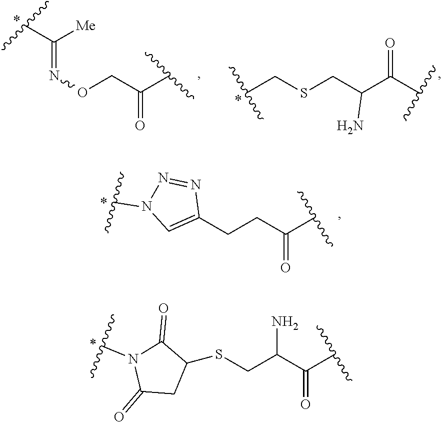 [SMILES: *C(=O)CON=C(*)C ; *CSCC(N)C(*)=O ; *C(=O)CCc1cn(*)nn1 ; *C(=O)C(N)CSC1CC(=O)N(*)C1=O]

and;

wherein * denotes a point of attachment of group E to group D.

In one embodiment, E is E3. Preferably R²⁰ is H and R²¹ is ethylene.

In one embodiment, E is E4. Preferably R²⁰ is methyl and R²¹ is methylene.

In one embodiment, E is E93. In one embodiment E is E94.

In one embodiment, E is E97. Preferably R²¹ is methylene.

In one embodiment D is D2 wherein R¹⁵ is

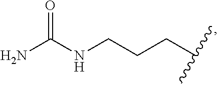 [SMILES: *CCCNC(N)=O]

### R¹⁶ is

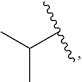 [SMILES: *C(C)C]

R³² is C₆-alkylene and E is E4 wherein R²⁰ is methyl and R²¹ is methylene.

In one embodiment G is selected from FFRK, GFLG and FKRL.

In one embodiment G is

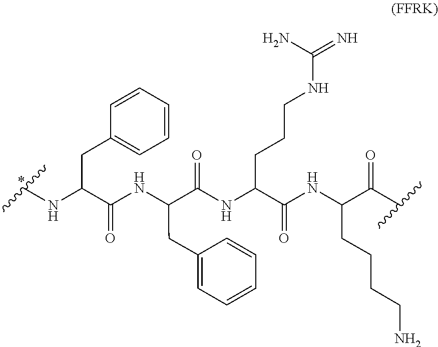 [SMILES: *NC(Cc1ccccc1)C(=O)NC(Cc1ccccc1)C(=O)NC(CCCNC(=N)N)C(=O)NC(CCCCN)C(*)=O]

wherein * denotes a point of attachment of group G to group E.

In one embodiment G is absent.

In one embodiment, R⁴ is CH₂OH.

In one embodiment, R⁶ is OH. In another embodiment, R⁶ is OR¹².

In one embodiment, R⁷ is OR¹². In another embodiment, R⁷ is OH.

In one embodiment R⁷ is OR¹² and R⁶ is OH.

In one embodiment R⁶ is OR¹² and R⁷ is OH.

Alternatively it is preferred that R⁶ and R⁷ are both OR¹².

Alternatively it is preferred that R⁷ is H and R⁶ is OR¹².

In one embodiment, R⁸ is C₁-C₁₅ alkyl. In one embodiment R⁸ is C₁-C₁₅ alkyl having a straight or branched carbon chain containing no double bonds, triple bonds, oxygen atoms or aryl groups. In one embodiment R⁸ is C₁₀ to C₁₄ alkyl. In one embodiment, R⁸ is C₁₀ to C₁₄ alkyl having a straight or branched carbon chain containing no double bonds, triple bonds, oxygen atoms or aryl groups.

In one embodiment R⁸ is C₁₄ alkyl. In one embodiment, R⁸ is C₁₄ alkyl having a straight carbon chain containing no double bonds, triple bonds, oxygen atoms or aryl groups.

In one embodiment R⁸ is C₁-C₁₅ alkyl having a straight or branched carbon chain containing no double bonds, triple bonds, oxygen atoms or aryl groups, R⁷ is OR¹² and R⁶ is OH.

In one embodiment R¹¹ is alkyl, preferably lower alkyl.

In one embodiment, R¹² is C₆-C₃₀ acyl having a straight carbon chain containing no double bonds, triple bonds, oxygen atoms, aryl groups and which is unsubstituted.

In one embodiment R¹² is C₁₈-C₂₆ acyl. In one embodiment, R¹² is C₁₈-C₂₆ acyl having a straight carbon chain containing no double bonds, triple bonds, oxygen atoms, aryl groups and which is unsubstituted.

More preferably R¹² is C₂₆ acyl. In one embodiment, R¹² is C₂₆ acyl having a straight carbon chain containing no double bonds, triple bonds, oxygen atoms, aryl groups and which is unsubstituted.

In one embodiment R¹² is acyl having a straight carbon chain from 18 to 26 carbon atoms long and having an optionally substituted chain terminating aryl group. More preferably R¹² is C₁₁ acyl having an optionally substituted chain terminating aryl group.

Still more preferably the optionally substituted aryl group is phenyl, optionally substituted with a halogen, e.g. a fluorine, e.g. the optionally substituted aryl group is p-fluorophenyl. In one embodiment R¹² is C₆-C₃₀ acyl having a straight carbon chain with an optionally substituted chain terminating aryl group.

The conjugates for use in the combination of the invention include a peptide antigen (J).

In one embodiment J is a peptide antigen that contains within its sequence one or more epitopes that bind to MHC molecules and induce T cell responses.

In one embodiment the peptide antigen incorporates two or more epitopes from either a single protein, or from several proteins.

The peptide antigen may be derived from a protein which is a suitable target for prophylactic or therapeutic vaccines. The protein may originate from a tumour, virus, bacteria or other source.

A peptide antigen “derived from” a target protein is to be understood herein as to comprise a contiguous amino acid sequence selected from the target protein, which, while preserving its antigenic properties, may be modified by deletion or substitution of one or more amino acids, by extension at the N- and/or C-terminus with additional amino acids or functional groups.

In one embodiment, J is a peptide antigen from a tumour, virus or bacteria. In one embodiment, J is a peptide antigen from a cancerous tumour.

In one embodiment, the peptide antigen has a length of 8 to 100 amino acids, or from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 70, 80 or 90 to 100 amino acids.

In one embodiment, the peptide antigen has a length of 18 to 100 amino acids. In one embodiment, the peptide antigen has a length of 20-45 amino acids. In one embodiment, the peptide antigen has a length of 20-35 amino acids.

In one embodiment, the peptide antigen is a neo-antigen. As used herein, a “neo-antigen” is a tumour antigen that arises from a tumour-specific mutation(s) which alters the amino acid sequence of genome-encoded proteins. Neo-antigens can be identified by whole-genome sequencing elucidating all, or nearly all, mutated neo-antigens that are uniquely present in a cancer (or cancerous tumour) of an individual patient. This collection of mutated neo-antigens may be analysed to identify a specific, optimised subset of mutated new-epitopes for use as an antigen source for the development of personalised cancer vaccines. Methods to identify such neo-antigens are described in WO2014/168874, which is incorporated herein by reference.

A person skilled in the art would be able to select an appropriate peptide antigen for the particular treatment contemplated.

In one embodiment J comprises one or more peptides derived from antigens expressed by the tumour to be treated with the combination.

Preferably J is selected from the group consisting of: AMLGTHTMEV (SEQ ID NO:1), MLGTHTMEV (SEQ ID NO:2), EAAGIGILTV (SEQ ID NO:3), AAGIGILTV (SEQ ID NO:4), AADHRQLQLSISSCLQQL (SEQ ID NO:5), AAGIGILTVILGVL (SEQ ID NO:6), AARAVFLAL (SEQ ID NO:7), ACDPHSGHFV (SEQ ID NO:8), ACYEFLWGPRALVETS (SEQ ID NO:9), ADHRQLQLSISSCLQQL (SEQ ID NO:10), AEEAAGIGILT (SEQ ID NO:11), AEEAAGIGIL (SEQ ID NO:12), AELVHFLLL (SEQ ID NO:13), AELVHFLLLKYRAR (SEQ ID NO:14), AEPINIQTW (SEQ ID NO:15), AFLPWHRLF (SEQ ID NO:16), AGATGGRGPRGAGA (SEQ ID NO:17), ALCRWGLLL (SEQ ID NO:18), ALDVYNGLL (SEQ ID NO:19), ALFDIESKV (SEQ ID NO:20), ALGGHPLLGV (SEQ ID NO:21), ALIHHNTHL (SEQ ID NO:22), ALKDVEERV (SEQ ID NO:23), ALLAVGATK (SEQ ID NO:24), ALLEIASCL (SEQ ID NO:25), ALNFPGSQK (SEQ ID NO:26), ALPYWNFATG (SEQ ID NO:27), ALSVMGVYV (SEQ ID NO:28), ALWPWLLMAT (SEQ ID NO:29), ALWPWLLMA (SEQ ID NO:30), ALYVDSLFFL (SEQ ID NO:31), ANDPIFVVL (SEQ ID NO:32), APPAYEKLSAEQ (SEQ ID NO:33), APRGPHGGAASGL (SEQ ID NO:34), APRGVRMAV (SEQ ID NO:35), ARGPESRLL (SEQ ID NO:36), ASGPGGGAPR (SEQ ID NO:37), ATGFKQSSKALQRPVAS (SEQ ID NO:38), AVCPWTWLR (SEQ ID NO:39), AWISKPPGV (SEQ ID NO:40), AYVCGIQNSVSANRS (SEQ ID NO:41), CATWKVICKSCISQTPG (SEQ ID NO:42), CEFHACWPAFTVLGE (SEQ ID NO:43), CLSRRPWKRSWSAGSCPGMPHL (SEQ ID NO:44), CMTWNQMNL (SEQ ID NO:45), CQWGRLWQL (SEQ ID NO:46), CTACRWKKACQR (SEQ ID NO:47), DPARYEFLW (SEQ ID NO:48), DTGFYTLHVIKSDLVNEEATGQFRV (SEQ ID NO:49), DVTFNIICKKCG (SEQ ID NO:50), EAAGIGILTV (SEQ ID NO:51), EADPTGHSY (SEQ ID NO:52), EAFIQPITR (SEQ ID NO:53), EDLTVKIGDFGLATEKSRWSGSHQFEQLS (SEQ ID NO:54), EEAAGIGILTVI (SEQ ID NO:55), EEKLIVVLF (SEQ ID NO:56), EFYLAMPFATPM (SEQ ID NO:57), EGDCAPEEK (SEQ ID NO:58), EIIYPNASLLIQN (SEQ ID NO:59), EKIQKAFDDIAKYFSK (SEQ ID NO:60), ELTLGEFLKL (SEQ ID NO:61), ELVRRILSR (SEQ ID NO:62), ESRLLEFYLAMPF (SEQ ID NO:63), ETVSEQSNV (SEQ ID NO:64), EVDPASNTY (SEQ ID NO:65), EVDPIGHLY (SEQ ID NO:66), EVDPIGHVY (SEQ ID NO:67), EVISCKLIKR (SEQ ID NO:68), EVYDGREHSA (SEQ ID NO:69), EYLQLVFGI (SEQ ID NO:70), EYLSLSDKI (SEQ ID NO:71), EYSKECLKEF (SEQ ID NO:72), EYVIKVSARVRF (SEQ ID NO:73), FIASNGVKLV (SEQ ID NO:74), FINDEIFVEL (SEQ ID NO:75), FLDEFMEGV (SEQ ID NO:76), FLEGNEVGKTY (SEQ ID NO:77), FLFLLFFWL (SEQ ID NO:78), FLIIWQNTM (SEQ ID NO:79), FLLHHAFVDSIFEQWLQRHRP (SEQ ID NO:80), FLLLKYRAREPVTKAE (SEQ ID NO:81), FLTPKKLQCV (SEQ ID NO:82), FLWGPRALV (SEQ ID NO:83), FMNKFIYEI (SEQ ID NO:84), FMVEDETVL (SEQ ID NO:85), FPSDSWCYF (SEQ ID NO:86), FRSGLDSYV (SEQ ID NO:87), FSWAMDLDPKGA (SEQ ID NO:88), GARGPESRLLEFYLAMPFATPMEAELARRSLAQDAPPL (SEQ ID NO:89), GDNQIMPKAGLLIIV (SEQ ID NO:90), GELIGILNAAKVPAD (SEQ ID NO:91), GFKQSSKAL (SEQ ID NO:92), GLASFKSFLK (SEQ ID NO:93), GLCTLVAML (SEQ ID NO:94), GLPPDVQRV (SEQ ID NO:95), GLYDGMEHLI (SEQ ID NO:96), GRAMLGTHTMEVTVY (SEQ ID NO:97), GVALQTMKQ (SEQ ID NO:98), GVGSPYVSRLLGICL (SEQ ID NO:99), AKFVAAWTLKAAA (SEQ ID NO:100), GVLLKEFTVSGNILTIRLT (SEQ ID NO:101), GVLVGVALI (SEQ ID NO:102), GVYDGREHTV (SEQ ID NO:103), HLFGYSWYK (SEQ ID NO:104), HLIRVEGNLRVE (SEQ ID NO:105), HLSTAFARV (SEQ ID NO:106), HLYQGCQVV (SEQ ID NO:107), HQQYFYKIPILVINK (SEQ ID NO:108), HTMEVTVYHR (SEQ ID NO:109), IALNFPGSQK (SEQ ID NO:110), IGRIAECILGMNPSR (SEQ ID NO:111), IISAVVGIL (SEQ ID NO:112), ILAKFLHWL (SEQ ID NO:113), ILDSSEEDK (SEQ ID NO:114), ILDTAGREEY (SEQ ID NO:115), ILHNGAYSL (SEQ ID NO:116), ILSRDAAPLPRPG (SEQ ID NO:117), ILTVILGVL (SEQ ID NO:118), IMDQVPFFS (SEQ ID NO:119), IMDQVPFSV (SEQ ID NO:120), IMIGVLVGV (SEQ ID NO:121), INKTSGPKRGKHAWTHRLRE (SEQ ID NO:122), ISGGPRISY (SEQ ID NO:123), ISPNSVFSQWRVVCDSLEDYD (SEQ ID NO:124), ISQAVHAAHAEINEAGR (SEQ ID NO:125), ITDQVPFSV (SEQ ID NO:126), ITKKVADLVGF (SEQ ID NO:127), KASEKIFYV (SEQ ID NO:128), KAVYNFATM (SEQ ID NO:129), KCDICTDEY (SEQ ID NO:130), KEFTVSGNILT (SEQ ID NO:131), KEFTVSGNILTI (SEQ ID NO:132), KELEGILLL (SEQ ID NO:133), KHAWTHRLRERKQLVVYEEI (SEQ ID NO:134), KIFGSLAFL (SEQ ID NO:135), KIFSEVTLK (SEQ ID NO:136), KIFYVYMKRKYEAM (SEQ ID NO:137), KIFYVYMKRKYEAMT (SEQ ID NO:138), KILDAVVAQK (SEQ ID NO:139), KINKNPKYK (SEQ ID NO:140), KISQAVHAAHAEINEAGRESIINFEKLTEWT (SEQ ID NO:141), KKLLTQHFVQENYLEY (SEQ ID NO:142), KMDAEHPEL (SEQ ID NO: 143), KNCEPVVPNAPPAYEKLSAE (SEQ ID NO:144), KRYFKLSHLQMHSRKH (SEQ ID NO:145), KSSEKIVYVYMKLNYEVMTK (SEQ ID NO:146), KTWGQYWQV (SEQ ID NO:147), KVAELVHFL (SEQ ID NO:148), KVHPVIWSL (SEQ ID NO:149), KVLEYVIKV (SEQ ID NO:150), KYDCFLHPF (SEQ ID NO:151), KYVGIEREM (SEQ ID NO:152), LAALPHSCL (SEQ ID NO:153), LAAQERRVPR (SEQ ID NO:154), LAGIGILTV (SEQ ID NO:155), LAMPFATPM (SEQ ID NO:156), LGFKVTLPPFMRSKRAADFH (SEQ ID NO:157), LGPGRPYR (SEQ ID NO:158), LHHAFVDSIF (SEQ ID NO:159), LIYRRRLMK (SEQ ID NO:160), LKEFTVSGNILTIRL (SEQ ID NO:161), LKLSGVVRL (SEQ ID NO:162), LLANGRMPTVLQCVN (SEQ ID NO:163), LLDGTATLRL (SEQ ID NO:164), LLEFYLAMPFATPM (SEQ ID NO: 165), LLEFYLAMPFATPMEAELARRSLAQ (SEQ ID NO: 166), LLFGLALIEV (SEQ ID NO:167), LLGATCMFV (SEQ ID NO: 168), LLGPGRPYR (SEQ ID NO:169), LLGRNSFEV (SEQ ID NO:170), LLKYRAREPVTKAE (SEQ ID NO: 171), LLLDDLLVSI (SEQ ID NO: 172), LLLLTVLTV (SEQ ID NO:173), LLWSFQTSA (SEQ ID NO:174), LLYKLADLI (SEQ ID NO:175), LMLQNALTTM (SEQ ID NO: 176), LPAVVGLSPGEQEY (SEQ ID NO:177), LPHSSSHWL (SEQ ID NO:178), LPRWPPPQL (SEQ ID NO:179), LPSSADVEF (SEQ ID NO:180), LSHLQMHSRKH (SEQ ID NO: 181), LSRLSNRLL (SEQ ID NO: 182), LTDLQPYMRQFVAHL (SEQ ID NO:183), LWWVNNQSLPVSP (SEQ ID NO:184), LYATVIHDI (SEQ ID NO:185), LYSACFWWL (SEQ ID NO:186), LYVDSLFFL (SEQ ID NO: 187), MEVDPIGHLY (SEQ ID NO:188), MIAVFLPIV (SEQ ID NO: 189), MIFEKHGFRRTTPP (SEQ ID NO:190), MKLNYEVMTKLGFKVTLPPF (SEQ ID NO:191), MLAVISCAV (SEQ ID NO:192), MLLAVLYCL (SEQ ID NO:193), MLMAQEALAFL (SEQ ID NO: 194), MPFATPMEA (SEQ ID NO:195), MPREDAHFIYGYPKKGHGHS (SEQ ID NO:196), MSLQRQFLR (SEQ ID NO:197), MVKISGGPR (SEQ ID NO:198), NLVPMVATV (SEQ ID NO:199), NPPSMVAAGSVVAAV (SEQ ID NO:200), NSIVKSITVSASG (SEQ ID NO:201), NSNHVASGAGEAAIETQSSSSEEIV (SEQ ID NO:202), NSQPVWLCL (SEQ ID NO:203), NTYASPRFK (SEQ ID NO:204), NYARTEDFF (SEQ ID NO:205), NYKRCFPVI (SEQ ID NO:206), NYNNFYRFL (SEQ ID NO:207), PDTRPAPGSTAPPAHGVTSA (SEQ ID NO:208), PFATPMEAELARR (SEQ ID NO:209), PGSTAPPAHGVT (SEQ ID NO:210), PGTRVRAMAIYKQ (SEQ ID NO:211), PGVLLKEFTVSGNILTIRLTAADHR (SEQ ID NO:212), PLLENVISK (SEQ ID NO:213), PLPPARNGGL (SEQ ID NO:214), PLQPEQLQV (SEQ ID NO:215), PLTSIISAV (SEQ ID NO:216), PRALAETSYVKVLEY (SEQ ID NO:217), PVTWRRAPA (SEQ ID NO:218), PYYFAAELPPRNLPEP (SEQ ID NO:219), QCSGNFMGF (SEQ ID NO:220), QCTEVRADTRPWSGP (SEQ ID NO:221), QGAMLAAQERRVPRAAEVPR (SEQ ID NO:222), QGQHFLQKV (SEQ ID NO:223), QLAVSVILRV (SEQ ID NO:224), QNILLSNAPLGPQFP (SEQ ID NO:225), QQITKTEV (SEQ ID NO:226), QRPYGYDQIM (SEQ ID NO:227), QYSWFVNGTF (SEQ ID NO:228), RAGLQVRKNK (SEQ ID NO:229), REPFTKAEMLGSVIR (SEQ ID NO:230), REPVTKAEML (SEQ ID NO:231), RIAECILGM (SEQ ID NO:232), RKVAELVHFLLLKYR (SEQ ID NO:233), RKVAELVHFLLLKYRA (SEQ ID NO:234), RLLEFYLAMPFA (SEQ ID NO:235), RLLQETELV (SEQ ID NO:236), RLMKQDFSV (SEQ ID NO:237), RLPRIFCSC (SEQ ID NO:238), RLSSCVPVA (SEQ ID NO:239), RLVDDFLLV (SEQ ID NO:240), RMPEAAPPV (SEQ ID NO:241), RMPTVLQCVNVSVVS (SEQ ID NO:242), RNGYRALMDKS (SEQ ID NO:243), RNGYRALMDKSLHVGTQCALTRR (SEQ ID NO:244), RPGLLGASVLGLDDI (SEQ ID NO:245), RPHVPESAF (SEQ ID NO:246), RQKRILVNL (SEQ ID NO:247), RSDSGQQARY (SEQ ID NO:248), RTKQLYPEW (SEQ ID NO:249), RVIKNSIRLTL (SEQ ID NO:250), RVRFFFPSL (SEQ ID NO:251), RYQLDPKFI (SEQ ID NO:252), SAFPTTINF (SEQ ID NO:253), SAWISKPPGV (SEQ ID NO:254), SAYGEPRKL (SEQ ID NO:255), SEIWRDIDF (SEQ ID NO:256), SELFRSGLDSY (SEQ ID NO:257), SESIKKKVL (SEQ ID NO:258), SESLKMIF (SEQ ID NO:259), SFSYTLLSL (SEQ ID NO:260), SHETVIIEL (SEQ ID NO:261), SIINFEKL (SEQ ID NO:262), SLADTNSLAV (SEQ ID NO:263), SLFEGIDIYT (SEQ ID NO:264), SLFPNSPKWTSK (SEQ ID NO:265), SLFRAVITK (SEQ ID NO:266), SLGWLFLLL (SEQ ID NO:267), SLLMWITQC (SEQ ID NO:268), SLLMWITQCFLPVF (SEQ ID NO:269), SLLQHLIGL (SEQ ID NO:270), SLPYWNFATG (SEQ ID NO:271), SLSKILDTV (SEQ ID NO:272), SLYKFSPFPL (SEQ ID NO:273), SLYSFPEPEA (SEQ ID NO:274), SNDGPTLI (SEQ ID NO:275), SPRWWPTCL (SEQ ID NO:276), SPSSNRIRNT (SEQ ID NO:277), SQKTYQGSY (SEQ ID NO:278), SRFGGAVVR (SEQ ID NO:279), SSALLSIFQSSPE (SEQ ID NO:280), SSDYVIPIGTY (SEQ ID NO:281), SSKALQRPV (SEQ ID NO:282), SSPGCQPPA (SEQ ID NO:283), STAPPVHNV (SEQ ID NO:284), SVASTITGV (SEQ ID NO:285), SVDYFFVWL (SEQ ID NO:286), SVSESDTIRSISIAS (SEQ ID NO:287), SVYDFFVWL (SEQ ID NO:288), SYLDSGIHF (SEQ ID NO:289), SYLQDSDPDSFQD (SEQ ID NO:290), TFPDLESEF (SEQ ID NO:291), TGRAMLGTHTMEVTVYH (SEQ ID NO:292), TLDSQVMSL (SEQ ID NO:293), TLDWLLQTPK (SEQ ID NO:294), TLEEITGYL (SEQ ID NO:295), TLMSAMTNL (SEQ ID NO:296), TLNDECWPA (SEQ ID NO:297), TLPGYPPHV (SEQ ID NO:298), TLYQDDTLTLQAAG (SEQ ID NO:299), TMKQICKKEIRRLHQY (SEQ ID NO:300), TMNGSKSPV (SEQ ID NO:301), TPRLPSSADVEF (SEQ ID NO:302), TSCILESLFRAVITK (SEQ ID NO:303), TSEKRPFMCAY (SEQ ID NO:304), TSYVKVLHHMVKISG (SEQ ID NO:305), TTEWVETTARELPIPEPE (SEQ ID NO:306), TVSGNILTIR (SEQ ID NO:307), TYACFVSNL (SEQ ID NO:308), TYLPTNASL (SEQ ID NO:309), TYYRPGVNLSLSC (SEQ ID NO:310), VAELVHFLL (SEQ ID NO:311), VFGIELMEVDPIGHL (SEQ ID NO:312), VGQDVSVLFRVTGALQ (SEQ ID NO:313), VIFSKASSSLQL (SEQ ID NO:314), VISNDVCAQV (SEQ ID NO:315), VLDGLDVLL (SEQ ID NO:316), VLFYLGQY (SEQ ID NO:317), VLHWDPETV (SEQ ID NO:318), VLLKEFTVSG (SEQ ID NO:319), VLLQAGSLHA (SEQ ID NO:320), VLPDVFIRCV (SEQ ID NO:321), VLPDVFIRC (SEQ ID NO:322), VLRENTSPK (SEQ ID NO:323), VLYRYGSFSV (SEQ ID NO:324), VPGVLLKEFTVSGNILTIRLTAADHR (SEQ ID NO:325), VPLDCVLYRY (SEQ ID NO:326), VRIGHLYIL (SEQ ID NO:327), VSSFFSYTL (SEQ ID NO:328), VVLGVVFGI (SEQ ID NO:329), VVPCEPPEV (SEQ ID NO:330), VVVGAVGVG (SEQ ID NO:331), VYFFLPDHL (SEQ ID NO:332), WEKMKASEKIFYVYMKRK (SEQ ID NO:333), WLPFGFILI (SEQ ID NO:334), WNRQLYPEWTEAQRLD (SEQ ID NO:335), WQYFFPVIF (SEQ ID NO:336), WRRAPAPGA (SEQ ID NO:337), YACFVSNLATGRNNS (SEQ ID NO:338), YFSKKEWEKMKSSEKIVYVY (SEQ ID NO:339), YLEPGPVTA (SEQ ID NO:340), YLEPGPVTV (SEQ ID NO:341), YLNDHLEPWI (SEQ ID NO:342), YLQLVFGIEV (SEQ ID NO:343), YLSGANLNL (SEQ ID NO:344), YLVPQQGFFC (SEQ ID NO:345), YMDGTMSQV (SEQ ID NO:346), YMIMVKCWMI (SEQ ID NO:347), YRPRPRRY (SEQ ID NO:348), YSVYFNLPADTIYTN (SEQ ID NO:349), YSWRINGIPQQHTQV (SEQ ID NO:350), YVDFREYEYY (SEQ ID NO:351), YYWPRPRRY (SEQ ID NO:352), IMDQVPFFS (SEQ ID NO:353), SVDYFFVWL (SEQ ID NO:354), ALFDIESKV (SEQ ID NO:355), NLVPMVATV (SEQ ID NO:356) and GLCTLVAML (SEQ ID NO:357), SVASTITGV (SEQ ID NO:358), VMAGDIYSV (SEQ ID NO:359), ALADGVQKV (SEQ ID NO:360), LLGATCMFV (SEQ ID NO:361), SVFAGVVGV (SEQ ID NO:362), ALFDGDPHL (SEQ ID NO:363), YVDPVITSI (SEQ ID NO:364), STAPPVHNV (SEQ ID NO:365), LAALPHSCL (SEQ ID NO:366), SQDDIKGIQKLYGKRS (SEQ ID NO:367), FLPSDFFPSV (SEQ ID NO:368), FLPSDFFPSV (SEQ ID NO:369), TLGEFLKLDRERAKN (SEQ ID NO:370), TFSYVDPVITSISPKYGMET (SEQ ID NO:371), AMTQLLAGV (SEQ ID NO:372), KVFAGIPTV (SEQ ID NO:373), AIIDGVESV (SEQ ID NO:374), GLWHHQTEV (SEQ ID NO:375), NLDTLMTYV (SEQ ID NO:376), KIQEILTQV (SEQ ID NO:377), LTFGDVVAV (SEQ ID NO:378), TMLARLASA (SEQ ID NO:379), IMDQVPFSV (SEQ ID NO:380), MHQKRTAMFQDPQERPRKLPQLCTELQTTIHD (SEQ ID NO:381), LPQLCTELQTTI (SEQ ID NO:382), HDIILECVYCKQQLLRREVY (SEQ ID NO:383), KQQLLRREVYDFAFRDLCIVYRDGN (SEQ ID NO:384), RDLCIVYRDGNPYAVCDKCLKFYSKI (SEQ ID NO:385), DKCLKFYSKISEYRHYCYSLYGTTL (SEQ ID NO:386), HYCYSLYGTTLEQQYNKPLCDLLIR (SEQ ID NO:387), YGTTLEQQYNKPLCDLLIRCINCQKPLCPEEK (SEQ ID NO:388), RCINCQKPLCPEEKQRHLDKKQRFHNIRGRWT (SEQ ID NO:389), DKKQRFHNIRGRWTGRCMSCCRSSRTRRETQL (SEQ ID NO:390), MHGDTPTLHEYMLDLQPETTDLYCYEQLNDSSEEE (SEQ ID NO:391), LYCYEQLNDSSEEEDEIDGPAGQAEPDRAHYNIVT (SEQ ID NO:392), GQAEPDRAHYNIVTFCCKCDSTLRLCVQSTHVDIR (SEQ ID NO:393), TLRLCVQSTHVDIRTLEDLLMGTLGIVCPICSQKP (SEQ ID NO:394), ALPFGFILV (SEQ ID NO:395), TLADFDPRV (SEQ ID NO:396), IMDQVPFSV (SEQ ID NO:397), SIMTYDFHGA (SEQ ID NO:398), AQYIKANSKFIGITEL (SEQ ID NO:399), FLYDDNQRV (SEQ ID NO:400), YLIELIDRV (SEQ ID NO:401), NLMEQPIKV (SEQ ID NO:402), FLAEDALNTV (SEQ ID NO:403), ALMEQQHYV (SEQ ID NO:404), ILDDIGHGV (SEQ ID NO:405), KLDVGNAEV (SEQ ID NO:406), TFEFTSFFY (SEQ ID NO:407), SWPDGAELPF (SEQ ID NO:408), GILGFVFTL (SEQ ID NO:409), ILRGSVAHK (SEQ ID NO:410) SVYDFFVWLKFFHRTCKCTGNFA (SEQ ID NO:411), DLAQMFFCFKELEGW (SEQ ID NO:412), AVGALEGPRNQDWLGVPRQL (SEQ ID NO:413) and RAHYNIVTF (SEQ ID NO:414).

Still more preferably J is selected from the group consisting of:

IMDQVPFSV, YLEPGPVTV, LAGIGILTV, YMDGTMSQV, SIINFEKL, ISQAVHAAHAEINEAGR, KISQAVHAAHAEINEAGRESIINFEKLTEWT, KAVYNFATM, MLMAQEALAFL, SLLMWITQC, GARGPESRLLEFYLAMPFATPMEAELARRSLAQDAPPL, VPGVLLKEFTVSGNILTIRLTAADHR, ESRLLEFYLAMPF, SLLMWITQCFLPVF, ILHNGAYSL, GVGSPYVSRLLGICL, AKFVAAWTLKAAA, IMDQVPFFS, SVDYFFVWL, ALFDIESKV, NLVPMVATV and GLCTLVAML.

Alternatively more preferably J is selected from the group consisting of:

SVASTITGV, VMAGDIYSV, ALADGVQKV, LLGATCMFV, SVFAGVVGV, ALFDGDPHL, YVDPVITSI, STAPPVHNV, LAALPHSCL, SQDDIKGIQKLYGKRS, FLPSDFFPSV, FLPSDFFPSV, TLGEFLKLDRERAKN, TFSYVDPVITSISPKYG MET, AMTQLLAGV, KVFAGIPTV, AIIDGVESV, GLWHHQTEV, NLDTLMTYV, KIQEILTQV, LTFGDVVAV, TMLARLASA, IMDQVPFSV, MHQKRTAMFQDPQERPRKLPQLCTELQTTIHD, LPQLCTELQTTI, HDIILECVYCKQQLLRREVY, KQQLLRREVYDFAFRDLCIVYRDGN, RDLCIVYRDGNPYAVCDKCLKFYSKI, DKCLKFYSKISEYRHYCYSLYGTTL, HYCYSLYGTTLEQQYNKPLCDLLIR, YGTTLEQQYNKPLCDLLIRCINCQKPLCPEEK, RCINCQKPLCPEEKQRHLDKKQRFHNIRGRWT, DKKQRFHNIRGRWTGRCMSCCRSSRTRRETQL, MHGDTPTLHEYMLDLQPETTDLYCYEQLNDSSEEE, LYCYEQLNDSSEEEDEIDGPAGQAEPDRAHYNIVT, GQAEPDRAHYNIVTFCCKCDSTLRLCVQSTHVDIR, TLRLCVQSTHVDIRTLEDLLMGTLGIVCPICSQKP, ALPFGFILV, TLADFDPRV, IMDQVPFSV, SIMTYDFHGA, FLYDDNQRV, YLIELIDRV, NLMEQPIKV, FLAEDALNTV, ALMEQQHYV, ILDDIGHGV, and KLDVGNAEV.

The combinations of the invention also include a TLR-9 agonist. In one embodiment the TLR-9 agonist is a CpG oligodeoxynucleotide. CpG oligonucleotides suitable for use in the combinations of the invention include Types A, B and C.

Also suitable for use in the combinations of the invention are two 3′-3′ linked single-stranded CpG oligodeoxynucleotides, a molecule comprising two loops each containing three CpG motifs connected with a double stranded stem, and dual function CpG-conjugates with STAT2 inhibitors as described in Adamus et al. 2018 (herein incorporated by reference).

In one embodiment, the TLR-9 agonist is an oligodeoxynucleotide comprising a core sequence comprising a hexamer with a central unmethylated cytosine-phosphate-guanine (CpG) moiety, having a general formula RRCGYY; wherein R represents a purine; C represents a cytosine; G represents a guanine; and Y represents a pyrimidine.

Examples of such agents are discussed in Klinman 1996.

In one embodiment the TLR-9 agonist is an unmethylated single-stranded CpG oligodeoxynucleotide of about 15 to about 30 bases. In one embodiment the bases of the TLR-9 agonist are linked via natural phosphodiester linkages. In one embodiment the bases of the TLR-9 agonist are linked via phosphorothioate linkages.

In one embodiment the TLR-9 agonist is a duplex comprising an oligodeoxynucleotide sequence together with its complimentary 3′ palindrome.

In one embodiment the TLR-9 agonist comprises GTCGTT.

In one embodiment the TLR-9 agonist comprises the sequence 5′-TCGTCGTTTGTCGTTTGTCGTT-3′ linked via a phosphorothioate backbone (ODN 7909).

In one embodiment the TLR-9 agonist comprises the sequence 5′-TGACTGTGAACGTTCGAGATGA-3 linked via a phosphorothioate backbone (1018 Dynavax).

In one embodiment the TLR-9 agonist comprises the sequence 5′-TCG*AACG*TTCG*-X-G*CT TG*CAAG*CT-5′ linked via a phosphorothioate backbone, where G* represents 2′-deoxy-7-deazaguanosine and X is a glycerol linker (IMO-2125 Idera).

In one aspect the invention relates to a pharmaceutical composition comprising a combination of a TLR-9 agonist and a conjugate of any one of Formulae (I), (II), (III), (IV) or (V), and a pharmaceutically acceptable carrier or excipient.

In one aspect the invention relates to a vaccine comprising a combination of a TLR-9 agonist and a conjugate of any one of Formulae (I), (II), (III), (IV) or (V), and a pharmaceutically acceptable carrier or excipient.

In one aspect the invention relates to a kit comprising at least two unit dosage forms (A) and (B), wherein (A) comprises a TLR-9 agonist and (B) comprises a conjugate of any one of Formulae (I), (II), (III), (IV) or (V) or a pharmaceutically acceptable salt thereof; and optionally (C) instructions for the simultaneous, sequential or separate administration of (A) and (B).

Examples of suitable pharmaceutically acceptable carriers and excipients are described in Remington's Pharmaceutical Sciences 18th Ed., Gennaro, ed. (Mack Publishing Co. 1990).

Numerous pharmaceutically acceptable carriers and excipients are approved by relevant government regulatory agencies. Examples of pharmaceutically acceptable carriers and excipients include sterile liquids such as water and oils, including animal, vegetable, synthetic or petroleum oils, saline solutions, aqueous dextrose and glycerol solutions, starch glucose, lactose, sucrose, gelatine, sodium stearate, glycerol monostearate, sodium chloride, propylene glycol, ethanol, wetting agents, emulsifying agents, binders, dispersants, thickeners, lubricants, pH adjusters, solubilizers, softening agents, surfactants and the like.

The pharmaceutical compositions and vaccines as described herein are suitable for administration parenterally, intratumourally, or peritumourally.

The pharmaceutical composition or vaccine of the invention is formulated so as to allow the active agents within to be bioavailable upon administration to a subject. For parenteral administration, the compositions and vaccines can be formulated as known in the art, for example, in a sterile aqueous solution, suspension or emulsion that optionally comprises other substances, such as salt or glucose, but not limited thereto. The pharmaceutical composition may include one or more of the following carriers or excipients: sterile diluents such as water, saline solution, Ringer's solution, isotonic sodium chloride, fixed oils such as squalene, mineral oil, mannide monooleate, cholesterol, mono or di-glycerides, polyethylene glycols, glycerine, propylene glycol, antibacterial agents such as methyl paraben or benzyl alcohol, antioxidants such as ascorbic acid or sodium bisulfite, and chelating agents such as ethylenediaminetetraacetic acid, buffers such as acetates, citrates or phosphates.

The pharmaceutical composition or vaccine of the invention may also include components such as, but not limited to, water-in-oil emulsions, liposomes, micellar components, microparticles, biodegradable microcapsules and liposomes.

The compositions and vaccines described herein can be presented in unit dosage form and can be prepared by any of the methods well known in the art of pharmacy. The term “unit dosage form” means a single dose wherein all active and inactive ingredients are combined in a suitable system, such that the patient or person administering the drug can open a single container or package with the entire dose contained therein and does not have to mix any components together from two or more containers or packages. Any examples of unit dosage forms are not intended to be limiting in any way, but merely to represent typical examples in the pharmacy arts of unit dosage forms.

### 5.4 Uses of the Combinations of the Invention

As discussed above, the inventors have surprisingly found that a combination of a TLR-9 agonist and a glycolipid-peptide conjugate of any one of Formulae (I), (II), (III), (IV) or (V) is extremely effective at treating solid tumour cancers, and can also induce regression at tumours distal to the injected site.

The combination of glycolipid-peptide conjugate and TLR-9 agonist is far more effective than either treatment administered alone, demonstrating a surprising synergistic effect between the two components of the combination.

As shown in Example 5, in the treatment of established EG7.OVA tumours, combining CpG 1826 with conjugate CI-191 (containing an OVA-derived epitope expressed in these tumours as a unique antigen) induced durable complete regression in the majority of animals, and in all animals (8 of 8) when the administration of CpG was delayed. This observation was seen in both the treated (injected) tumours and distal tumours, highlighting the significant abscopal effect (FIG. 1).

In contrast, treatment with CpG failed to induce complete regression in any animals, and conjugate CI-191 alone induced complete regression in only 1 of 8 animals (FIG. 1B).

The anti-tumour impact of combination therapy is therefore more than additive when compared to use of the single agents.

Example 6 demonstrates use of the combinations of the invention in the treatment of TC-1 tumours. Again, tumour regression was seen at both the injected site and the distant site, the latter being impacted via the abscopal effect (FIG. 2).

The combination of the invention was also effective in Example 7, where the conjugate alone showed no activity. When glycolipid-peptide conjugate vaccine CI-181 was used to target a series of defined neoepitopes in B16-F10 melanoma, no obvious anti-tumour activity was observed, suggesting the vaccine was incapable of inducing a response of sufficient size or potency to reach the threshold for clinical impact (FIG. 3). Treatment with CpG 1826 alone also had only a marginal impact. Despite this, when the conjugate and CpG 1826 were combined, significant anti-tumour activity was observed. Again this shows that the components present in the combination of the invention act synergistically to drive a large immune response.

This synergistic effect was shown again in the B16-OVA model (FIG. 4), although in this case the tumours were naturally more immunogenic, so the anti-tumour activity seen with treatment was greater. In fact, full regressions were observed in the majority of animals injected with CpG 1826 and conjugate CI-181.

Accordingly, the data provided herein demonstrates that the combinations of the invention can be used to treat cancers associated with tumour formation.

The data demonstrates an anti-tumour effect against both the injected tumour and distal tumours (an abscopal effect). Where tumours can be accessed but not easily resected, intratumoural administration of the combination of the invention could induce regression of the tumour mass. In addition, the combination of the invention may result in regression of inaccessible and/or inoperable tumours, including metastatic tumours.

The data also demonstrates an enhanced anti-tumour response following intratumoural administration of the combination, providing a systemic protective effect.

Accordingly, the combination of a TLR-9 agonist and a conjugate of any one of Formulae (I), (II), (III), (IV) or (V) may provide an effective cancer treatment, contributing to an improvement in survival rates and quality of life.

In one embodiment the conjugate of any one of Formulae (I), (II), (III), (IV) or (V) is as defined herein for any of the embodiments set out for and encompassed within the conjugate aspects of the invention.

In one aspect the invention provides a method of inducing regression of a cancerous tumour in a subject, comprising administering to the subject a therapeutically effective amount of a combination of a TLR-9 agonist and a conjugate of any one of Formula (I), (II), (III), (IV) or (V) or a pharmaceutically acceptable salt thereof, wherein the combination is administered by intratumoural or peritumoural injection.

In one embodiment, the cancerous tumour regresses by at least 20, 30, 40, 50, 60, 70, 80, 90, 95 or 99%. In one embodiment, the cancerous tumour regresses by 100%.

In another aspect the invention relates to a method of treating cancer in a subject, the method comprising administering to the subject a therapeutically effective amount of a combination of a TLR-9 agonist and a conjugate of any one of Formulae (I), (II), (III), (IV) or (V), or a pharmaceutically acceptable salt thereof; wherein the combination is administered by intratumoural or peritumoural injection to a cancerous tumour.

In another aspect the invention relates to a method of reducing the incidence of recurrence of cancer in a subject, the method comprising administering to the subject a therapeutically effective amount of a combination of a TLR-9 agonist and a conjugate of any one of Formulae (I), (II), (III), (IV) or (V), or a pharmaceutically acceptable salt thereof;

wherein the combination is administered by intratumoural or peritumoural injection to a cancerous tumour.

In the methods of the invention, the combination of the invention is injected intratumourally (injection directly into a tumour) or peritumourally (injection into the peritumoral region of a tumour).

In one embodiment, the peritumoral region of a tumour is defined as the region surrounding the tumour out to a distance from the tumoural boundary. In one embodiment, the peritumoral region is the region extending 25 mm from the tumoural boundary. In one embodiment, the peritumoral region is the region extending 10 mm from the tumoural boundary. In one embodiment, the peritumoral region is the region extending 5 mm from the tumoural boundary. In one embodiment, the peritumoral region is the region extending 2.5 mm from the tumoural boundary. In one embodiment, the peritumoral region is the region extending 1 mm from the tumoural boundary.

In one embodiment, the combination is administered by intratumoural injection.

Administration of the combination of the invention means that the two agents present in the combination are administered to the subject at the same time or within a time interval such that the effects of each agent on the subject overlap synergistically, to provide a more than additive effect.

In one embodiment, the TLR-9 agonist and a conjugate of any one of Formulae (I), (II), (III), (IV) or (V) are administered simultaneously by intratumoural or peritumoural injection, preferably intratumoural injection.

In one embodiment, the TLR-9 agonist and a conjugate of any one of Formulae (I), (II), (III), (IV) or (V) are administered sequentially by intratumoural or peritumoural injection, preferably intratumoural injection.

The inventors have found that the anti-tumour effect is enhanced when the glycolipid-peptide conjugate is administered prior to the TLR-9 agonist. In the mouse models used, a 6 hour delay was found to be highly effective. As will be appreciated by a person skilled in the art, the administration regime that provides the best results will vary depending in the cancerous tumour and the subject being treated.

In one embodiment, the TLR-9 agonist is injected intratumourally or peritumourally (preferably intratumourally) after a conjugate of any one of Formulae (I), (II), (III), (IV) or (V) is injected intratumourally or peritumourally, preferably intratumourally.

In one embodiment, the TLR-9 agonist is injected intratumourally or peritumourally (preferably intratumourally) about 3 to about 9 hours after a conjugate of any one of Formulae (I), (II), (III), (IV) or (V) is injected intratumourally or peritumourally, preferably intratumourally.

In one embodiment, the TLR-9 agonist is injected intratumourally or peritumourally (preferably intratumourally) about 6 hours after a conjugate of any one of Formulae (I), (II), (III), (IV) or (V) is injected intratumourally or peritumourally, preferably intratumourally.

In one embodiment, administration of the combination of a TLR-9 agonist and a conjugate of Formulae (I), (II), (III), (IV) or (V) generates an abscopal effect.

The appropriate ratios of each component of the combination to be administered will depend on the type of cancer being treated and other clinical considerations. In one embodiment, the TLR-9 agonist and a conjugate of Formulae (I), (II), (III), (IV) or (V) are administered in a molar ratio of between 1:10 to 10:1, preferably between 5:1 to 1:5, more preferably between 2:1 and 1:2.

In one embodiment, the TLR-9 agonist is administered at a dose of 0.3 to 200 micrograms per immunisation. In one embodiment, the conjugate of Formulae (I), (II), (III), (IV) or (V) is administered at a dose of 0.1 to 200 micrograms per immunisation.

In one embodiment, the cancer is selected from the group comprising a carcinoma, sarcoma and lymphoma. In one embodiment, the tumour is a primary tumour. In one embodiment, the tumour is a metastatic tumour. In one embodiment the selected from the group comprising melanoma, lung cancer, cervical cancer, ovarian cancer, uterine cancer, breast cancer, liver cancer, gastric cancer, colon cancer, prostate cancer, pancreatic cancer, kidney cancer, bladder cancer, brain cancer, Merkel cell carcinoma, T-cell lymphoma, squamous cell carcinoma and hepatocellular carcinoma.

The above embodiments of the invention also apply to the following statements mutatis mutandis.

The invention also provides a combination of a TLR-9 agonist and a conjugate of any one of Formulae (I), (II), (III), (IV) or (V) or a pharmaceutically acceptable salt thereof, for intratumoural or peritumoural administration for treating cancer.

The invention also provides a TLR-9 agonist and a conjugate of any one of Formulae (I), (II), (III), (IV) or (V) or a pharmaceutically acceptable salt thereof, for use in a combination therapy for cancer, wherein the TLR-9 agonist and conjugate of any one of Formula (I), (II), (III), (IV) or (V) or a pharmaceutically acceptable salt thereof are administered intratumourally to a cancerous tumour.

In one embodiment the TLR-9 agonist and conjugate of any one of Formulae (I), (II), (III), (IV) or (V) or a pharmaceutically acceptable salt thereof or administered sequentially to the cancerous tumour.

The invention also provides a use of a TLR-9 agonist and a conjugate of any one of Formulae (I), (II), (III), (IV) or (V) or a pharmaceutically acceptable salt thereof, in the manufacture of a medicament for the treatment of cancer, wherein the TLR-9 agonist and conjugate are to be administered intratumourally or peritumourally to a cancerous tumour.

The invention also provides a use of a TLR-9 agonist and a conjugate of any one of Formulae (I), (II), (III), (IV) or (V) or a pharmaceutically acceptable salt thereof, in the manufacture of a medicament for the treatment of cancer, wherein the TLR-9 agonist and conjugate, are formulated for intratumourally or peritumourally administration to a cancerous tumour.

In one embodiment, the medicament is formulated for sequential, simultaneous administration of the TLR-9 agonist and conjugate.

Various aspects of the invention will now be illustrated in non-limiting ways by reference to the following examples.

### 6. EXAMPLES

### 6.1 Materials and Methods

Mice

C57BL/6 were bred and maintained at the BRU, Malaghan Institute of Medical Research, New Zealand. All mice used were 6-12 weeks of age and littermates of the same sex were randomly assigned to experimental groups. Experimental procedures were approved by the Animal Ethics Committee at Victoria University and carried out in accordance with guidelines of Victoria University of Wellington, New Zealand.

Solubilization of Conjugates for Biological Studies

Solubilization of α-GalCer and α-GalCer-peptide conjugates was achieved by lyophilizing the samples in the presence of aqueous sucrose, L-histidine and Tween 20 as previously described for the solubilization of α-GalCer (Giaccone, 2002). Typically, all conjugates were reconstituted in water then further diluted in PBS for intratumoural administration. For CpG 1836, reconstitution was in endotoxin-free water and then further dilution was done in PBS, while the HPV E7 peptide was reconstituted in DMSO and diluted to a working concentration in PBS.

Subcutaneous Challenge with Tumour Cells

Tumour cell lines were maintained in complete Iscove's modified Dulbecco's medium (cIMDM), comprising IMDM (Invitrogen, Auckland, New Zealand) supplemented with 1% penicillin-streptomycin from (Invitrogen), and 5% fetal bovine serum (FBS; Sigma-Aldrich). Media was filter sterilized and stored at 4° C. for a maximum of 14 days. For the TC1 cell line, complete RPMI medium was used, where RPMI medium (Invitrogen) was supplemented with 1% penicillin-streptomycin, 10% FBS, 1% glutamax, and 1% sodium pyruvate (all Invitrogen). For tumour challenge, EG7.OVA cells were washed three times in complete medium, and 10⁶ tumour cells were injected subcutaneously into the flanks of mice. For B16.F10 or B16.OVA, 3×10⁵ cells were injected subcutaneously For TC-1 tumours 10⁵ cells were injected subcutaneously. Tumour size was measured using calipers, and recorded as the product of two bisecting diameters. Mice were culled when tumour sizes reached exceeded 200 mm².

Intratumoural Treatment

Intratumoural treatment was initiated once tumour size reached ˜36 mm². Mice were anaesthetized using isofluorane, and once under sedation each of the conjugates (at doses and regimens specified in the figure descriptions) was injected intratumourally in a total volume of 40 μl using a 300 μl insulin needle. The specific injection technique involved gentle insertion of the needle into a single site for each injection, but changing the site for each treatment day. The plunger was depressed slowly such that liquid was released gradually over 5 seconds, the needle was then held inside injection site for 20 seconds after ejection of fluid and then slowly removed to prevent creation of a vacuum.

Synthesis of Glycolipid-Peptide Conjugates: General Synthesis Methods

Anhydrous solvents were obtained commercially. Air-sensitive reactions were carried out under Ar. Thin layer chromatography (TLC) was performed on aluminium sheets coated with 60 F254 silica. Flash column chromatography was performed on Reveleris® silica cartridges (38.6 μm) or SiliCycle® silica gel (40-63 μm). NMR spectra were recorded on a Bruker 500 MHz spectrometer. ¹H NMR spectra were referenced to tetramethylsilane at 0 ppm (internal standard) or to residual solvent peak (CHCl₃ 7.26 ppm, CHD₂OD 3.31 ppm, CHD₂(SO)CD₃ 2.50 ppm). ¹³C NMR spectra were referenced to tetramethylsilane at 0 ppm (internal standard) or to the deuterated solvent peak (CDCl₃ 77.0 ppm, CD₃OD 49.0 ppm, (CD3)₂SO 39.5 ppm). CDCl₃—CD₃OD solvent mixtures were always referenced to the methanol peak. High resolution electrospray ionization (ESI) mass spectra were undertaken on a Waters Q-TOF Premier™ Tandem Mass spectrometer fitted with a Waters 2795 HPLC. Semi-preparative HPLC and synthetic purity HPLC data were obtained on an Agilent 1100 system and peak identity was confirmed by LCMS on an Agilent 1260 HPLC with an Agilent 6130 single quadrupole mass spectroscopic detector using ESI. Each of these latter two systems was coupled to a Dionex Corona Ultra RS CAD as required.

Solubilization of Conjugates for Biological Studies

Solubilization of α-GalCer-peptide conjugates was achieved by lyophilizing the samples in the presence of aqueous sucrose, L-histidine and Tween 20 as previously described for the solubilization of α-GalCer (Giaccone, 2002). Typically, all conjugates were reconstituted in water then further diluted in PBS for intratumoural administration.

TLR-9 Agonist

Class B CpG 1826 (5′ TCC ATG ACG TTC CTG ACG TT 3′) was custom synthesised by Trilink Biotechnologies (San Diego, Calif., USA, 0-4100).

### Example 1—Synthesis of Peptides for Conjugation

N-Terminal Aminooxyacetyl-Peptides for Peptide Conjugations

Iterative Fmoc-SPPS for each peptide was performed on an automated Tribute (Protein technologies Inc., Arizona, USA) peptide synthesizer using the following reagents for each step, Fmoc protected amino acids (Fmoc-AA-OH, 5 mmol), the coupling agent HATU (4.8 mmol) and N-methylmorpholine (1.0 mmol) as the base. Acetyl capping, for unreacted N-terminus amine groups, was performed with acetic anhydride solution in DMF (20%, v/v). Deprotection of the Fmoc group was performed in a solution of piperidine in DMF (20%, v/v). Specific parameters for amino acid couplings are as follows i) Treatment of resin with 20% (v/v) piperidine in DMF (4 mL) for 2×7 minutes as a Fmoc protecting group deprotection step; ii) Washing of resin with DMF (4 mL) for 4×30 seconds; iii) Pre-dissolve and 2 minutes activation time of Fmoc-AA-OH, HATU and N-methylmorpholine (2 mL), iv) 50 minutes amino acid coupling time at room temperature; v) Capping of unreacted resin using 20% (v/v) acetic anhydride in DMF (1 mL) for 1 minute; vi) Washing of resin with DMF (3 mL) for 2×30 seconds.

Cysteine residues were coupled manually using the following conditions to avoid racemisation (the completion of the reaction was checked by Kaiser test), Fmoc-Cys(Trt)-OH (5 mmol), HATU (4.8 mmol) and HOAt (4.8 mmol) as a coupling agents, 2,4,6-trimethylpyridine (collidine, 10 mmol) as the base and DCM and DMF (5 mL, 1:1 v/v) as the solvent. Double couplings were performed for 50 min each.

The N-terminal Boc-aminooxy acetic acid group was coupled manually use the following conditions (the completion of the reaction was checked by Kaiser test), Boc-AoAA-NHS (3 mmol), 2,4,6-trimethylpyridine (7 mmol), DMF (2 mL) with a 20 min coupling time.

Upon the completion of the peptide synthesis, the resin was washed with DMF (4×8 mL), CH₂Cl₂ (4×5 mL) and dried under vacuum prior to the cleavage. The peptidyl-resin was treated with TFA/iPr₃SiH/H₂O/EDT (ethanedithiol) (10 mL, 94:1:2.5:2.5 v/v/v/v) for 75 min at room temperature. Following evaporation of TFA by blowing nitrogen gas over the mixture, the peptide was precipitated in cold diethyl ether, isolated by centrifugation (4000 rpm, 3×10 min). The precipitated product was dissolved in acetonitrile:water (1:1 v/v) containing 0.1% TFA, and lyophilised to afford the crude peptide.

Chromatographic separations were performed using a Waters XTerra®MS C-18 column (5μ; 4.6×150 mm) and a linear gradient of 5-65% B in 60 min, ca. 1% B per min at a flow rate of 1.0 mL/min. Buffer A: H₂O containing 0.1% TFA (v/v); Buffer B: acetonitrile containing 0.1% TFA (v/v). Purity was determined by LCMS and product ID carried by ESI-MS. The following peptides were prepared:

Peptide 573, (AoAA)-FFRKGQAEPDRAHYNIVTFCCKCDS-OH, purity>98%; Calcd. Mass: 3009.3527 gmol⁻¹, found 1505.1 [M+2H]²⁺,1003.8 [M+3H]³⁺, 753.0 [M+4H]⁴⁺, 830.0 [M+5H]⁵⁺, 692.0 [M+6H]⁶⁺

Peptide 575, (AoAA)-FFRKKISQAVHAAHAEINEAGRESIINFEKLTEWT, purity>98%; Calcd. Mass: 4145.6143 gmol⁻¹, 1382.7 [M+3H]³⁺, 1037.2 [M+4H]⁴⁺, 830.0 [M+5H]⁵⁺, 602.7 [M+6H]⁶⁺

Peptide 600, (AoAA)-FFRK-FVVKAYLPVNESFAFTADLRSNTGGQA-OH, purity>98%; Calcd. Mass: 3554.9789 gmol⁻¹, found 1185.3 [M+3H]³⁺, 889.2 [M+4H]⁴⁺, 711.5.

Peptide 601, (AoAA)-FFRK-ANFESGKHKYRQTAMFTATMPPAVERL-OH, purity>98%; Calcd. Mass: 3734.2797 gmol⁻¹, found 933.9 [M+4H]⁴⁺, 747.3 [M+5H]⁵⁺, 623.4 [M+6H]⁶⁺, 534.4 [M+7H]⁷⁺

Peptide 602, (AoAA)-FFRK-TPPPEEAMPFEFNGPAQGDHSQPPLQV-OH, purity>98%; Calcd. Mass: 3569.9221 gmol⁻¹, found 1785.7 [M+2H]²⁺, 1190.8 [M+3H]³⁺, 893.3 [M+4H]⁴⁺, 714.9 [M+5H]⁵⁺.

Peptide 603, (AoAA)-FFRK-VVDRNPQFLDPVLAYLMKGLCEKPLAS-OH, purity>95%; Calcd. Mass: 3669.3289 gmol⁻¹, found 1224.2 [M+3H]³⁺, 918.3 [M+4H]⁴⁺, 734.8 [M+5H]⁵⁺, 612.3 [M+6H]⁶⁺ where 1468.6 represents [2M+5H]⁵⁺

Peptide 604, (AoAA)-FFRK-FRRKAFLHWYTGEAMDEMEFTEAESNM-OH, purity>95%; Calcd. Mass: 3979.4425 gmol⁻¹, found 1327.3 [M+3H]³⁺, 995.7 [M+4H]⁴⁺, 796.7 [M+5H]⁵⁺ where 1592.4 represents [2M+5H]⁵⁺ and 1137.8 represents [2M+7H]⁷⁺

Peptide 605, (AoAA)-FFRK-SSPDEVALVEGVQSLGFTYLRLKDNYM-OH, purity>98%; Calcd. Mass: 3684.1497 gmol⁻¹, found 1228.8 [M+3H]³⁺, 921.8 [M+4H]⁴⁺, 737.8 [M+5H]⁵⁺

Peptide 606, (AoAA)-FFRK-PKPDFSQLQRNILPSNPRVTRFHINWD-OH, purity>98%; Calcd. Mass: 3928.4419 gmol⁻¹, found 1309.9 [M+3H]³⁺, 982.8 [M+4H]⁴⁺, 786.5 [M+5H]⁵⁺, 655.5 [M+6H]⁶⁺, 562.0 [M+7H]⁷⁺.

Peptide 607, (AoAA)-FFRK-TAVITPPTTTTKKARVSTPKPATPSTD-OH, purity>98%; Calcd. Mass: 3419.8949 gmol⁻¹, found 1140.6 [M+3H]³⁺, 855.7 [M+4H]⁴⁺, 684.8 [M+5H]⁵⁺, 570.8 [M+6H]⁶⁺.

Peptide 608, (AoAA)-FFRK-STANYNTSHLNNDVWQIFENPVDWKEK-OH, purity>98%; Calcd. Mass: 3902.2223 gmol⁻¹, found 1301.3 [M+3H]³⁺, 976.4 [M+4H]⁴⁺, 781.3 [M+5H]⁵⁺, 651.3 [M+6H]⁶⁺ where 1561.8 represents [2M+5H]⁵⁺

Peptide 609, (AoAA)-FFRK-REGVELCPGNKYEMRRHGTTHSLVIHD-OH, purity>98%; Calcd. Mass: 3787.2601 gmol⁻¹, found 1262.9 [M+3H]³⁺, 947.5 [M+4H]⁴⁺, 758.2 [M+5H]⁵⁺, 632.0 [M+6H]⁶⁺, 541.9 [M+7H]⁷+, 474.3 [M+8H]⁸⁺.

Peptide 610, (AoAA)-FFRK-NIEGIDKLTQLKKPFLVNNKINKIENI-OH, purity>98%; Calcd. Mass: 3789.4469 gmol⁻¹, found 1264.0 [M+3H]³⁺, 948.2 [M+4H]⁴⁺, 758.8 [M+5H]⁵⁺, 632.5 [M+6H]⁶⁺, 542.3 [M+7H]⁷⁺ where 1516.6 represents [2M+5H]⁵⁺

Peptide 611, (AoAA)-FFRK-IPSGTTILNCFHDVLSGKLSGGSPGVP-OH, purity>98%; Calcd. Mass: 3305.7687 gmol⁻¹, found 1102.5 [M+3H]³⁺, 827.0 [M+4H]⁴⁺, 662.0 [M+5H]⁵⁺

Peptide 612, (AoAA)-FFRK-PSKPSFQEFVDWENVSPELNSTDQPFL-OH, purity>98%; Calcd. Mass: 3790.1285 gmol⁻¹, found 1267.6 [M+3H]³⁺+Na⁺, 947.96 [M+4H]⁴⁺.

Peptide 613, (AoAA)-FFRK-DSGSPFPAAVILRDALHMARGLKYLHQ-OH, purity>98%; Calcd. Mass: 3616.1726 gmol⁻¹, found 1205.9 [M+3H]³⁺, 904.8 [M+4H]⁴⁺, 724.0 [M+5H]⁵⁺, 603.6 [M+6H]⁶⁺, 517.5 [M+7H]⁷⁺.

Peptide 614, (AoAA)-FFRK-CGTAFFINFIAIYHHASRAIPFGTMVA-OH, purity>98%; Calcd. Mass: 3608.2105 gmol⁻¹, found 1804.6 [M+2H]²⁺, 1203.3 [M+3H]³⁺, 902.7 [M+4H]⁴⁺, 722.4 [M+5H]⁵⁺.

Peptide 615, (AoAA)-FFRK-EFKHIKAFDRTFANNPGPMVVFATPGM-OH, purity>98%; Calcd. Mass: 3675.2548 gmol⁻¹, found 1225.3 [M+3H]³⁺, 919.2 [M+4H]⁴⁺, 735.5 [M+5H]⁵⁺, 613.1 [M+6H]⁶⁺

Peptide 616, (AoAA)-FFRK-ECRITSNFVIPSEYWVEEKEEKQKLIQ-OH, purity>98%; Calcd. Mass: 3978.5024 gmol⁻¹, found 1326.3 [M+3H]³⁺, 995.0 [M+4H]⁴⁺, 796.2 [M+5H]⁵⁺, 663.6 [M+6H]⁶⁺

Peptide 617, (AoAA)-FFRK-NHSGLVTFQAFIDVMSRETTDTDTADQ-OH, purity>98%; Calcd. Mass: 3651.9353 gmol⁻¹, found 1826.35 [M+2H]²⁺, 1217.93 [M+3H]³⁺, 913.68 [M+4H]⁴⁺

Peptide 618, (AoAA)-FFRK-GRGHLLGRLAAIVGKQVLLGRKVVVVR-OH, purity>98%; Calcd. Mass: 3517.2881 gmol⁻¹, found 1173.26 [M+3H]³⁺, 880.17 [M+4H]⁴⁺, 704.35 [M+5H]⁵⁺, 587.13 [M+6H]⁶⁺, 503.45 [M+7H]⁷⁺

Peptide 619, (AoAA)-FFRK-SHCHWNDLAVIPAGVVHNWDFEPRKVS-OH, purity>98%; Calcd. Mass: 3766.2255 gmol⁻¹, found 1256.0 [M+3H]³⁺, 942.2 [M+4H]⁴⁺, 754.0 [M+5H]⁵⁺, 628.5 [M+6H]⁶⁺, 538.8 [M+7H]⁷⁺.

Peptide 620, (AoAA)-FFRK-GFSQPLRRLVLHVVSAAQAERLARAEE-OH, purity>98%; Calcd. Mass: 3656.1795 gmol⁻¹, found 1219.4 [M+3H]³⁺, 914.8 [M+4H]⁴⁺, 732.1 [M+5H]⁵⁺, 610.3 [M+6H]⁶⁺, where 943.5 represents [M+4H]⁴⁺+114 Da (due to trifluoroacetic acid)

### Example 2—Synthesis of the Glycolipid Linker (MaGC-PAB-CV-Non) for Conjugation

### 4-(N-((9H-Fluoren-9-yl)methoxycarbonyl)-L-valinyl-L-citrullinamido)benzyl 4-nitrophenyl carbonate (Fmoc-VC-PAB-pNP)

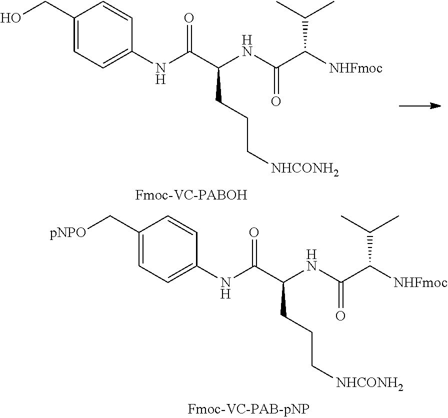

To a mixture of alcohol Fmoc-VC-PABOH (G. M. Dubowchik, 2002)(400 mg, 0.665 mmol) in DMF (6.0 mL) is added bis(4-nitrophenyl) carbonate (255 mg, 0.796 mmol) and i-Pr₂NEt (0.13 mL, 0.75 mmol). After stirring under Ar at rt for 18 h, the solvent is co-evaporated several times with toluene in a rotary evaporator. Purification by flash chromatography on silica gel (gradient elution, MeOH/CHCl₃=0:100 to 8:92) gave the title compound as a pale yellow solid (380 mg, 75%). ¹H NMR (500 MHz, d₆-DMSO) δ 0.85 (d, J=6.7 Hz, 3H), 0.88 (d, J=6.7 Hz, 3H), 1.33-1.49 (m, 2H), 1.56-1.64 (m, 1H), 1.67-1.74 (m, 1H), 1.95-2.02 (m, 1H), 2.91-2.97 (m, 1H), 2.99-3.06 (m, 1H), 3.92 (dd, J=7.2, 8.7 Hz, 1H), 4.20-4.26 (m, 2H), 4.28-4.33 (m, 1H), 4.39-4.43 (m, 1H), 5.24 (s, 2H), 5.39 (br s, 2H), 5.98 (br t, J=5.7 Hz, 1H), 7.30-7.33 (m, 2H), 7.36-7.42 (m, 5H), 7.54-7.57 (m, 2H), 7.63 (d, J=8.4 Hz, 2H), 7.70-7.74 (m, 2H), 7.87 (d, J=7.4 Hz, 2H), 8.10 (d, J=7.4 Hz, 1H), 8.29-8.32 (m, 2H), 10.10 (s, 1H); ¹³C NMR (126 MHz, d₆-DMSO) δ 18.3, 19.2, 26.8, 29.4, 30.5, 38.7, 46.8, 53.2, 60.2, 65.8, 70.3, 119.2, 120.1, 122.7, 125.4, 125.5, 127.2, 127.7, 129.46, 129.54, 139.4, 140.8, 143.8, 143.9, 145.3, 152.0, 155.4, 156.2, 159.1, 170.8, 171.4; HRMS-ESI: m/z calcd for C₄₀H₄₃N₆O₁₀ [M+H]+ 767.3041, found 767.3070.

### (2S,3S,4R)-2-(N-((9H-Fluoren-9-yl)methoxycarbonyl)-L-valinyl-L-citrullinyl-4-aminobenzyloxycarbonylamino)-1-(α-D-galactopyranosyloxy)-3-hydroxy-octadecan-4-yl hexacosanoate (MaGC-PAB-CV-Fmoc)

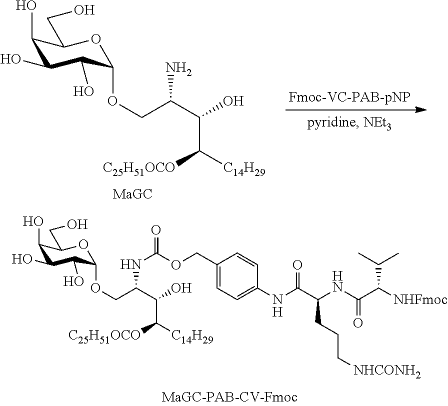

To a mixture of amine MaGC (R. J. Anderson, 2014)(73 mg, 0.085 mmol) and carbonate Fmoc-VC-PAB-pNP (93 mg, 0.12 mmol) in anhydrous pyridine (1.5 mL) under Ar is added Et₃N (16 μL, 12 mg, 0.11 mmol). After stirring at rt for 18 h, the mixture is concentrated to dryness and purified by column chromatography on silica gel (MeOH/CHCl₃=0:100 to 20:80) to afford the title compound as a white solid (66 mg, 52%). ¹H NMR (500 MHz, 2:3 CDCl₃/CD₃OD) δ 0.87-0.90 (m, 6H), 0.95-0.98 (m, 6H), 1.24-1.37 (m, 68H), 1.51-1.78 (m, 7H), 1.89-1.96 (m, 1H), 2.07-2.13 (m, 1H), 2.32-2.42 (m, 2H), 3.07-3.13 (m, 1H), 3.20-3.25 (m, 1H), 3.66-3.81 (m, 8H), 3.84-3.87 (m, 2H), 3.99 (d, J=6.7 Hz, 1H), 4.24 (t, J=6.9 Hz, 1H), 4.37 (dd, J=6.9, 10.5 Hz, 1H), 4.45 (dd, J=6.9, 10.5 Hz, 1H), 4.54 (dd, J=5.2, 8.6 Hz, 1H), 4.84 (d, J=3.7 Hz, 1H), 4.97-5.03 (m, 2H), 5.06-5.10 (m, 1H), 7.30-7.33 (m, 4H), 7.38-7.41 (m, 2H), 7.58 (d, J 10=8.1 Hz, 2H), 7.63-7.65 (m, 2H), 7.78 (d, J=7.6 Hz, 2H); ¹³C NMR (126 MHz, 2:1 CDCl₃/CD₃OD) δ14.3, 18.2, 19.4, 23.0, 25.5, 25.7, 26.7, 29.2, 29.6, 29.27, 29.74, 29.8, 29.93, 29.95, 29.98, 30.02, 30.05, 30.08, 30.10, 31.4, 32.3, 35.0, 39.4, 47.6, 52.7, 53.8, 61.2, 62.3, 66.8, 67.4, 68.4, 69.4, 70.2, 70.7, 71.0, 72.3, 75.1, 100.4, 120.3, 120.5, 125.40, 125.44, 127.5, 128.2, 129.1, 133.0, 138.2, 141.7, 144.2, 144.3, 157.1, 157.6, 161.1, 171.1, 173.2, 175.0; HRMS-ESI m/z calcd for C₈₄H₁₃₇N₆O₁₆ [M+H]⁺1486.0091, found 1486.0099.

### (2S,3S,4R)-2-(L-Valinyl-L-citrullinyl-4-aminobenzyloxycarbonylamino)-1-(α-D-galactopyranosyloxy)-3-hydroxy-octadecan-4-yl hexacosanoate (MaGC-PAB-CV-NH₂)

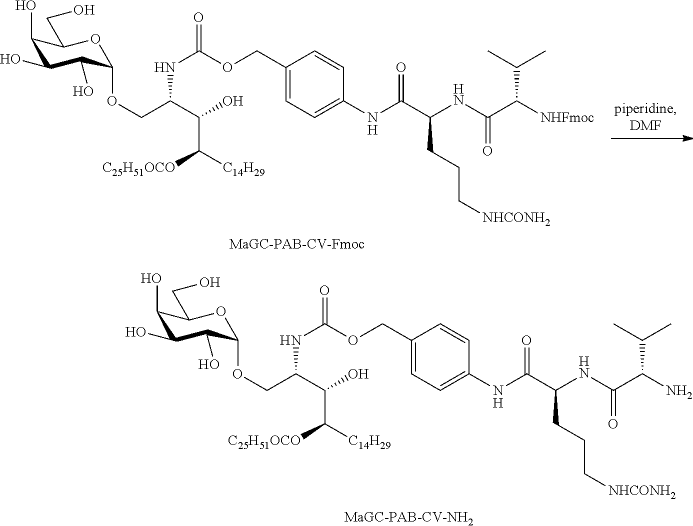

To an ice-cooled solution of MaGC-PAB-CV-Fmoc (66 mg, 0.044 mmol) in anhydrous DMF (2 mL) under Ar is added piperidine (0.20 mL, 2.0 mmol). After 5 min the mixture is warmed to rt and stirred for a further 30 min, before concentrating under high vacuum. Purification by flash chromatography on silica gel (MeOH/CHCl₃=0:100 to 60:40) gave the title compound as a white solid (45 mg, 81%). ¹H NMR (500 MHz, 2:1 CDCl₃/CD₃OD) δ0.87-0.91 (m, 9H), 1.00 (d, J=6.9 Hz, 3H), 1.23-1.35 (m, 68H), 1.49-1.77 (m, 7H), 1.87-1.94 (m, 1H), 2.07-2.13 (m, 1H), 2.32-2.39 (m, 2H), 3.10-3.16 (m, 1H), 3.21 (d, J=4.9 Hz, 1H), 3.24-3.29 (m, 1H), 3.65-3.80 (m, 8H), 3.85-3.87 (m, 2H), 4.57 (dd, J=5.3, 8.5 Hz, 1H), 4.85 (d, J=3.7 Hz, 1H), 4.92-4.99 (m, 2H), 5.10-5.15 (m, 1H), 7.33 (d, J=8.3 Hz, 2H), 7.56 (d, J=8.3 Hz, 2H); ¹³C NMR (75 MHz, 3:1 CDCl₃/CD₃OD) δ 14.1, 16.8, 19.5, 22.8, 25.2, 25.5, 26.4, 29.0, 29.4, 29.5, 29.6, 29.7, 29.8, 29.9, 30.0, 31.9, 32.1, 34.8, 39.2, 52.3, 53.1, 60.4, 62.1, 66.6, 68.2, 69.2, 70.0, 70.5, 70.6, 72.2, 74.9, 100.1, 120.3, 128.9, 132.9, 138.0, 156.8, 160.8, 171.1, 174.8, 175.7; HRMS-ESI m/z calcd for C₆₉H₁₂₇N₆O₄ [M+H]⁺ 1263.9410, found 1263.9419.

### (2S,3S,4R)-2-(N-(8-Oxononanoyl)-L-valinyl-L-citrullinyl-4-aminobenzyloxycarbonylamino)-1-(α-D-galactopyranosyloxy)-3-hydroxy-octadecan-4-yl hexacosanoate (MaGC-PAB-CV-Non)

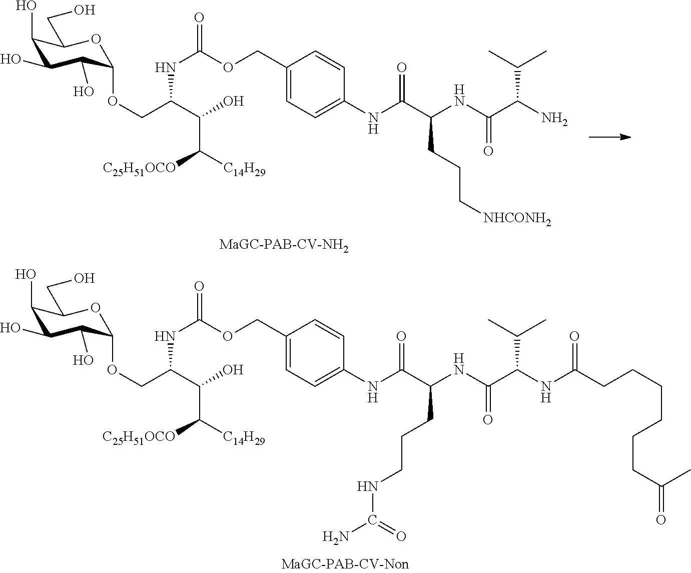

A DMF-solution (250 μL) containing 8-oxononanoic acid (2.2 mg, 12 μmol), i-Pr₂NEt (2.5 μL, 14 μmol) and 2-(1H-benzotriazol-1-yl)-1,1,3,3-tetramethyluronium hexafluorophosphate (HBTU) (2.6 mg, 6.8 μmol) is added to amine MaGC-PAB-CV-NH₂ (6.9 mg, 5.5 μmol) and the mixture is stirred at rt for 17 h. After concentrating under vacuum, the residue is purified by flash chromatography on silica gel (MeOH/CH₂Cl₂=8:92 to 20:80) and subsequently triturated with water to give the title compound as a white solid (6.6 mg, 85%). ¹H NMR (500 MHz, 2:1 CDCl₃/CD₃OD) δ 0.87-0.90 (m, 6H), 0.95-0.97 (m, 6H), 1.11-1.43 (m, 72H), 1.50-1.77 (m, 11H), 1.87-1.94 (m, 1H), 2.02-2.11 (m, 1H), 2.15 (s, 3H), 2.22-2.31 (m, 2H), 2.32-2.41 (m, 2H), 2.46 (t, J=7.3 Hz, 2H), 3.09-3.14 (m, 1H), 3.21-3.26 (m, 1H), 3.64-3.83 (m, 8H), 3.85-3.92 (m, 2H), 4.18 (d, J=7.3 Hz, 1H), 4.54 (dd, J=5.1, 8.5 Hz, 1H), 4.85 (d, J=3.7 Hz, 1H), 4.93-5.02 (m, 1H), 5.13 (d, J=12.2 Hz, 1H), 7.32 (d, J=8.2 Hz, 2H), 7.57 (d, J=8.2 Hz, 2H); ¹³C NMR (126 MHz, 2:1 CDCl₃/CD₃OD) δ14.21, 14.22, 18.5, 19.4, 23.0, 23.9, 25.3, 25.4, 25.7, 25.9, 26.7, 29.1, 29.3, 29.6, 29.69, 29.71, 29.8, 29.89, 29.91, 29.95, 29.98, 30.01, 30.04, 30.05, 30.06, 31.0, 32.3, 35.0, 36.4, 43.9, 52.6, 53.7, 59.4, 62.3, 66.8, 68.4, 69.4, 70.2, 70.7, 71.0, 72.3, 75.1, 100.4, 120.5, 129.1, 133.0, 157.1, 161.1, 171.0, 172.9, 175.0, 175.2, 211.4; HRMS-ESI m/z calcd for C₇₈H₁₄₀N₆NaO₁₆ [M+Na]⁺ 1440.0218, found 1440.0214.

### Example 3—Conjugate Manufacture by Oxime Ligation

General Method for Synthesis of Vaccines by Oxime Ligation

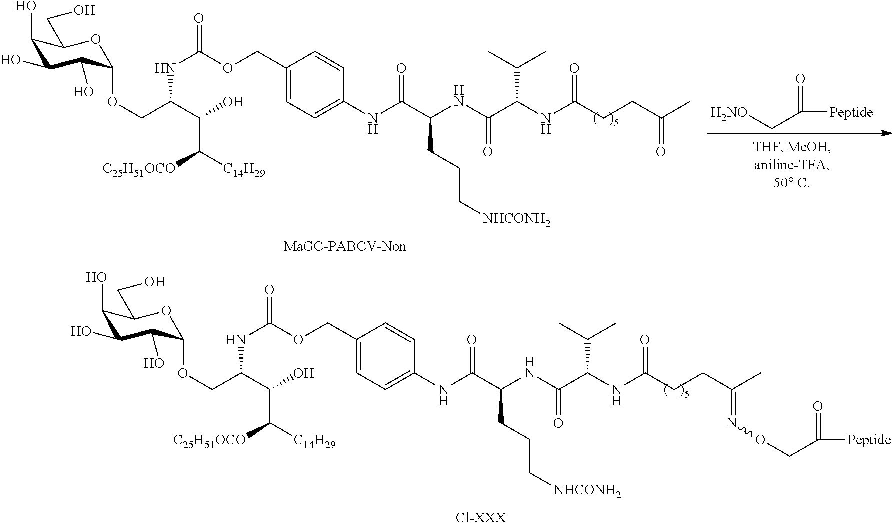

Aniline buffer (pH=4.1, 300 mM) is prepared by mixing freshly distilled aniline (5.5 mL) and TFA (3.9 mL) in MilliQ water, and making up to a total volume of 200 mL. THF is distilled from 2,4-dinitrophenylhydrazine.

A mixture of ketone MaGC-PABCV-Non (2.0 mg, 1.4 μmol) and N-terminal aminooxyacetylated peptide (1.5-2.0 equiv) is heated in 4:2:3 THF/MeOH/aniline buffer (600 μL) under an Ar atmosphere at 50° C. for 15-24 h until the ketone starting material is consumed (LCMS). Crude products containing cysteine residues are treated with aq TCEP-HCl (100 mg/mL, 10-20 equiv) before chromatographic purification. The product mixture is purified by preparative HPLC (Phenomenex Luna C18(2), 5 μm, 250×21 mm, 40° C., 20 mL/min; Mobile phase A=100:0.05 water/TFA; Mobile phase B=100:0.05 MeOH/TFA; 0-8 min: 70-100% B; 8-10 min: 100% B; 10-11 min: 100-70% B; 11-13 min: 700/B). Partial concentration (rotary evaporator) followed by lyophilization gives the conjugate product as a white solid. The following conjugates were prepared using the above method.

| | Amt | | | | | |
|---|---|---|---|---|---|---|
| | ketone | | | | | |
| | starting | | Yield of | Formula for m/z | Calcd | Found |
| Conjugate | material | Peptide | conjugate | calculation | m/z | m/z |
| CI-112 | 2.0 mg | Pep573 | 5.6 mg | C₂₀₈H₃₃₄N₄₄O₅₄S₃ | 2204.1947 | 2204.1953 |
| | | | (90%) | [M + 2H]²⁺ | | |
| CI-191 | 1.9 mg | Pep575 | 4.7 mg | C₂₆₄H₄₂₅N₅₉O₇₀ | 1386.5455 | 1386.5486 |
| | | | (64%) | [M + 4H]⁴⁺ | | |
| CI-130 | 2.0 mg | Pep612 | 5.9 mg | C₂₅₃H₃₉₁N₄₈O₆₈ | 1729.9537 | 1729.9521 |
| | | | (81%) | [M + 3H]³⁺ | | |
| CI-131 | 2.0 mg | Pep615 | 5.9 mg | C₂₄₉H₃₉₅N₅₁O₅₇S₂ | 1268.9755 | 1268.9772 |
| | | | (82%) | [M + 4H]⁴⁺ | | |
| CI-132 | 2.0 mg | Pep616 | 4.9 mg | C₂₅₉H₄₁₉N₅₂O₆₈S | 1792.6882 | 1792.6903 |
| | | | (64%) | [M + 3H]³⁺ | | |
| CI-133 | 2.0 mg | Pep600 | 5.6 mg | C₂₄₂H₃₈₇N₄₉O₆₁ | 1238.9672 | 1238.9667 |
| | | | (80%) | [M + 4H]⁴⁺ | | |
| CI-134 | 2.0 mg | Pep601 | 5.3 mg | C₂₄₆H₄₀₀N₅₄O₆₀S₂ | 1283.7338 | 1283.7365 |
| | | | (74%) | [M + 4H]⁴⁺ | | |
| CI-135 | 2.1 mg | Pep602 | 5.9 mg | C₂₄₀H₃₇₅N₄₈O₆₃S | 1656.5779 | 1656.5746 |
| | | | (81%) | [M + 3H]³⁺ | | |
| CI-136 | 2.1 mg | Pep603 | 4.7 mg | C₂₄₇H408N₄₉O₅₉S₂ | 1689.6625 | 1689.6604 |
| | | | (63%) | [M + 3H]³⁺ | | |
| CI-137 | 1.7 mg | Pep604 | 3.7 mg | C₂₅₇H₃₉₉N₅₃O₆₆S₃ | 1344.9664 | 1344.9652 |
| | | | (57%) | [M + 4H]⁴⁺ | | |
| CI-138 | 2.1 mg | Pep605 | 4.6 mg | C₂₄₅H₃₉₈N₄₈O₆₅S | 1271.2258 | 1271.2231 |
| | | | (62%) | [M + 4H]⁴⁺ | | |
| CI-139 | 2.1 mg | Pep606 | 6.6 mg | C₂₅₈H₄₁₄N₆₀O₆₁ | 1332.2784 | 1332.2798 |
| | | | (85%) | [M + 4H]⁴⁺ | | |
| CI-140 | 1.9 mg | Pep607 | 6.3 mg | C₂₃₀H₃₉₃N₄₈O₆₂ | 1606.6359 | 1606.6381 |
| | | | (97%) | [M + 3H]³⁺ | | |
| CI-141 | 2.1 mg | Pep613 | 5.7 mg | C₂₄₃H₃₉₈N₅₄O₅₇S | 1254.2406 | 1254.2417 |
| | | | (77%) | [M + 4H]⁴⁺ | | |
| CI-144 | 1.9 mg | Pep609 | 1.6 mg | C₂₄₂H₃₉₉N₅₉O₆₂S₂ | 1296.9832 | 1296.9839 |
| | | | (23%) | [M + 4H]⁴⁺ | | |
| CI-145 | 2.1 mg | Pep611 | 3.3 mg | C₂₂₇H₃₇₅N₄₆O₅₈S | 1568.5844 | 1568.5859 |
| | | | (47%) | [M + 3H]³⁺ | | |
| CI-146 | 2.0 mg | Pep614 | 3.0 mg | C₂₄₉H₃₈₉N₅₀O₅₄S₂ | 1669.2891 | 1669.2887 |
| | | | (42%) | [M + 3H]³⁺ | | |
| CI-147 | 2.1 mg | Pep619 | 2.3 mg | C₂₅₁H₃₉₁N₅₆O₅₉S | 1721.9679 | 1721.9684 |
| | | | (30%) | [M + 3H]³⁺ | | |
| CI-148 | 1.0 mg | Pep617 | 0.5 mg | C₂₃₆H₃₈₁N₅₀O₆₉S | 1683.9187 | 1683.9207 |
| | | | (14%) | [M + 3H]³⁺ | | |
| CI-149 | 1.0 mg | Pep618 | 2.6 mg | C₂₃₉H₄₂₀N₅₉O₅₀ | 1639.0712 | 1639.0731 |
| | | | (75%) | [M + 3H]³⁺ | | |
| CI-150 | 1.0 mg | Pep620 | 2.1 mg | C₂₄₁H₄₀₆N₅₈O₅₉ | 1264.2638 | 1264.2627 |
| | | | (59%) | [M + 4H]⁴⁺ | | |
| CI-179 | 1.9 mg | Pep608 | 4.0 mg | C₂₅₅H₃₉₆N₅₄O₆₈ | 1325.7297 | 1325.7308 |
| | | | (57%) | [M + 4H]⁴⁺ | | |
| CI-180 | 1.9 mg | Pep610 | 5.5 mg | C₂₅₂H₄₂₉N₅₃O₆₂ | 1297.5511 | 1297.5535 |
| | | | (78%) | [M + 4H]⁴⁺ | | |

CI-181 is a mixture of CI-130-C1-141, CI-144-CI-150, CI-179 and CI-180

### Example 4—Formulation of Conjugates

Conjugates of the invention were formulated similarly to reported methods for α-GalCer, as described by Giaccone et al. (Giaccone, 2002). For neoantigen conjugates CI-130-CI-141, CI-144-CI-150, CI-179, CI-180, each conjugate was dissolved in DMSO at 1.1 mM, and 4 μL of each solution added to one vial containing 837 μL of an aqueous solution of Tween 20 (7.5 mg), sucrose (83 mg) and L-histidine (11.2 mg). After mixing thoroughly, aliquots of 98 μL were portioned out and lyophilized with 0.5 mL water to give the formulated product (in 50 μg portions) as an equimolar mixture of all 21 neoantigen conjugates. Formulation of individual neoantigen conjugates was achieved by adding 8.92 μL of each DMSO solution to separate vials containing 89 μL of aqueous Tween 20/sucrose/L-histidine in the same proportions as above. The resulting homogenous solutions were lyophilized with 0.5 mL water to give the formulated conjugates (in 46-53 μg amounts). Conjugates CI-112 and CI-112 were formulated individually, in a similar manner.

### Example 5: Intratumoural Injection of CpG with a Glycolipid-Peptide Conjugate is Synergistic when Compared to Either Agent on its Own, Providing a Strong Antitumour Response with Abscopal Effect

To test the anti-tumour effect of combining intratumoural CpG with a glycolipid-peptide conjugate CI-191 containing a T cell epitope from a tumour-associated antigen, treatment with the conjugates alone or in combination was assessed in C57BL/6 mice subcutaneously engrafted on both flanks with EG7.OVA tumours, a T lymphoma expressing ovalbumin (OVA) as a model of a tumour-associated antigen. To test whether the treatment induced a systemic immune response with abscopal effect, the intratumoural treatment was restricted to tumours on one flank only. The dosing regimen for CpG 1826 consisted of three treatments four days apart, starting on day 6, where the compound was injected directly into the tumour mass on two consecutive days. Treatment with conjugate CI-191 (containing the OVA-derived epitope) was just once intratumourally on day 6. Two approaches to combined therapy were tested; one where the conjugate was delivered at the same time as the first CpG 1826, and one where the conjugate was administered first, and the initial CpG 1826 was delayed by 6 h. A control group of animals received vehicle administered with the same regimen as the delayed combination group. As shown in FIG. 1, combining treatment of CpG 1826 with a conjugate CI-191 induced durable complete regression in the majority of animals regardless of dosing schedule. Notably, complete regression was seen in all animals (8 of 8) when administration of CpG 1826 was after the conjugate. These powerful responses were seen at both the treated and distant sites, highlighting a significant abscopal effect. In contrast, treatment with CpG 1826 alone failed to induce complete regression in any animals, and the conjugate alone induced complete regression in only 1 of 8 animals. The anti-tumour impact of combination therapy is therefore more than additive when compared to the single agents.

### Example 6: Intratumoural Injection of CpG with a Glycolipid-Peptide Conjugate Induces Stronger Antitumour Immune Responses when Compared to CpG with Unconjugated Peptide and Glycolipid

Animals were engrafted with subcutaneous TC-1 tumours, a lung carcinoma expressing human papilloma virus (HPV) oncoproteins as a model of HPV-associated cancer. The tumours were on both flanks, and treatment was conducted on only one flank in order to test for abscopal effect. A group of animals were injected with a combination of CpG 1826 combined with a glycolipid-peptide conjugate (CI-112). A further group of animals were injected with peptide only to evaluate the impact of simply introducing a relevant antigen to the tumour. The molar dose of conjugate and peptide was kept consistent between the groups.

The peptide used (also contained within the conjugate, CI-112) was a known CD8⁺ T cell epitope from HPV E7 oncoprotein. As shown in FIG. 2, combining CpG 1826 with conjugate CI-112 induced a strong anti-tumour response, with a high percentage of complete regressions. This was seen at both the injected site and the distant site, which was impacted via the abscopal effect.

### Example 7: Intratumoural Injection of CpG with a Mixture of Glycolipid-Peptide Conjugates Induces Stronger Antitumour Immune Response than when CpG is Combined with α-GalCer

To further test the relevance of conjugating peptide to the NKT cell agonist, a B16-F10 melanoma tumour model was used where the peptide sequences of 21 different putative neoepitopes have been defined (Kreiter, 2015). Conjugate vaccine CI-181 was prepared as a mixture of 21 conjugates, each containing peptide for a different putative neoepitope. As shown in FIG. 3 intratumoural injection of CI-181 alone had no anti-tumour impact. Nor did injection of CpG 1826 alone. However, the combination of CpG 1826 and CI-181 had a more than additive effect on antitumour activity, with tumour onset delayed approximately 10 days (on average). This treatment was the only one to induce any complete regressions. This synergistic effect of combined treatment is agreement with the results obtained in EG7.OVA tumours in FIG. 1.

A similar effect was observed in the B16-OVA model (FIG. 4), although in this case the tumours were naturally more immunogenic, so the anti-tumour activity seen with treatment was greater.

### Example 8: Intratumoural Injection of CpG with Glycolipid-Peptide Conjugate Induces Anti-Tumour Immune Responses than can Prevent Re-Challenge with the Same Tumour

To assess whether the immune responses induced with CpG 1826 and conjugates have immunological memory, and so could prevent subsequent tumour onset of the same malignancy, animals that had completely rejected treated (and distal) tumours were re-challenged with the same tumour. As shown in FIG. 5, C57BL/6 mice that had rejected TC-1 tumours after intratumoural treatment with CpG 1826 and glycolipid-peptide conjugate CI-112 were completely resistant to re-challenge with TC-1. The combined treatment is therefore associated with strong immunological memory.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art. Each of such external documents is also specifically incorporated by reference herein.

### 7. INDUSTRIAL APPLICABILITY

The combinations, compositions, methods and uses of the invention find use in the treatment of cancer in a subject and/or in the reduction of the incidence of recurrence of cancer in a subject.

### 8. REFERENCES

Dubowchik, G. M. et al. (2002). Bioconjugate Chem., 13, 855-869.
Giaccone, P. et al. (2002). Clin. Cancer Res., 8(12), 3702-9.
Kreiter, S. et al. (2015). Nature, 520(7549), 692-696.
Anderson, R. J. et al. (2014). Nat. Chem. Biol., 10, 943-949.
Adamas, T. et al. (2018) Contemp Oncol (Pozn), 56-60.
Klinman, D. M. et al. (1996) PNAS 93(7) 2879-2883.

1-34. (canceled)

35. A combination of a TLR-9 agonist and a conjugate of Formula (I) or pharmaceutically acceptable salt thereof, 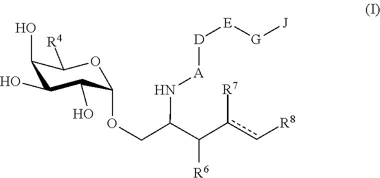
wherein:
A is a self-immolative linker group;
D is selected from the group consisting of: 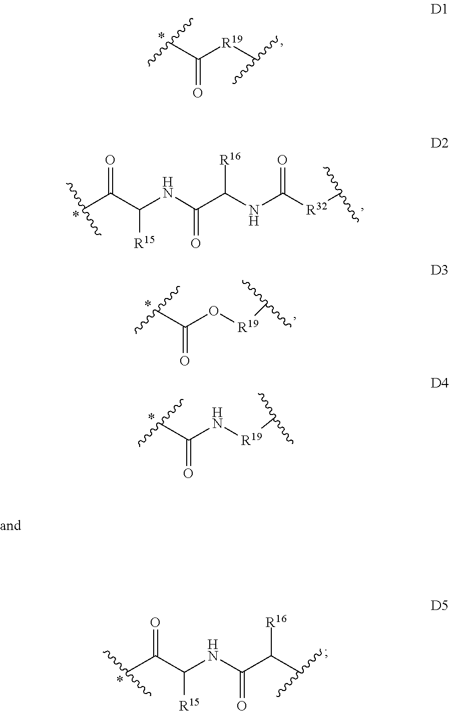
wherein * denotes a point of attachment of group D to group A;
R¹⁵ is a side chain of one of the following amino acids: L-lysine, L-citrulline, L-arginine, L-glutamine or L-threonine;
R¹⁶ is a side chain of a hydrophobic amino acid;
R¹⁹ is an alkylene group;
R³² is an alkylene group or an O-alkylene group wherein the O is attached to the carbonyl group of D2;
E is selected from the group consisting of: 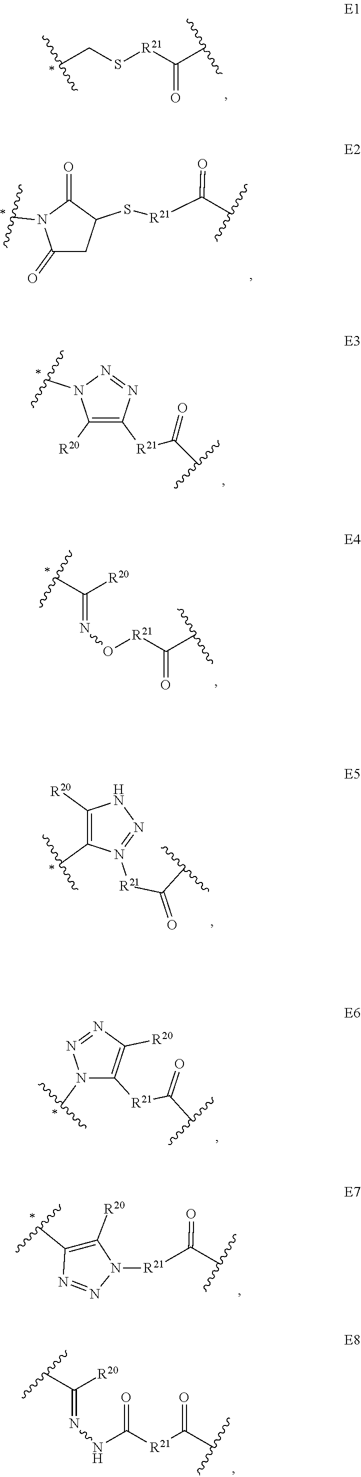 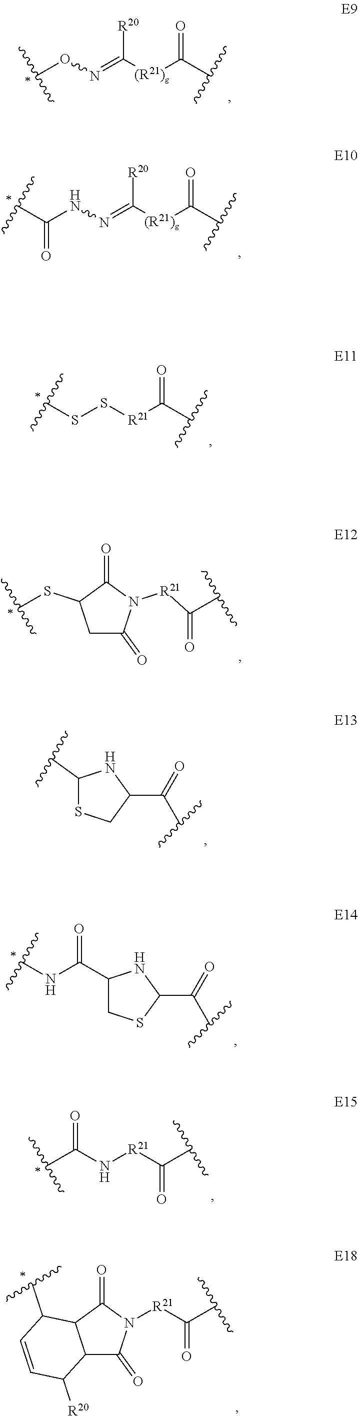 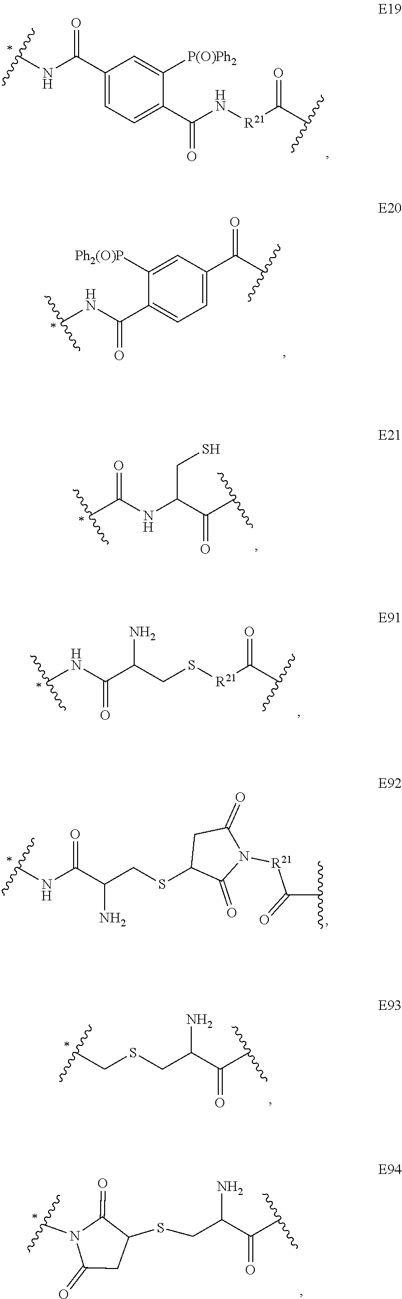 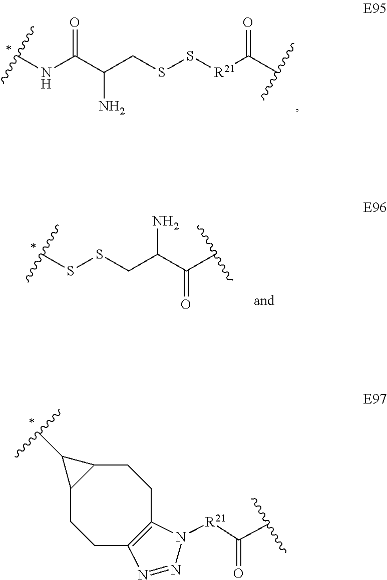
wherein * denotes a point of attachment of group E to group D;
R²⁰ is H or lower alkyl;
R²¹ is an alkylene group;
g is 0 when R²⁰ is H or g is 1 when R²⁰ is lower alkyl;
provided that E is E18 only when D is D1, D2 or D3 and provided that E is E1, E2, E3, E4, E5, E6, E7, E8, E9, E10, E11, E12, E13, E15, E20, E21, E93, E94 or E96 only when D is D1, D2, D3 or D4; and provided that E is E91, E92 or E95 only when D is D5 and provided that E is E97 only when D is D2;
G is absent or G is an amino acid sequence of up to 6 amino acids, attached through its N-terminus to group E and through its C-terminus to group J;
J is a peptide antigen, optionally substituted at its N and/or C-termini with up to 6 amino acids selected from the group of natural flanking residues for the antigen, and optionally terminated with NH₂ at the C-terminus so as to provide a C-terminal amide, and attached to group G through its N-terminus or, wherein G is absent, attached to group E through its N-terminus;
R⁴ is CH₃, CH₂OH, CH₂OCOR¹¹, CH₂OR¹¹, CH₂OSO₃H, CH₂SH, CH₂SR¹¹, CH₂SOR¹¹, CH₂SO₂R¹¹, CH₂PO₃H₂, CH₂OP(O)(OH)₂, CH₂OP(O)(OH)(OR¹¹), CH₂OP(O)(OR¹¹)₂, CO₂H, CH₂NHCOR¹¹, CH₂NHCO₂R¹¹, CH₂NHCONH₂, CH₂NHCONHR¹¹, CH₂NHCON(R¹¹)₂, CH₂N(R¹¹)₂, CH₂NHSO₂R¹¹;
R⁶ is OR¹², OH or H;
R⁷ is OR¹², OH or H; provided that at least one of R⁶ and R⁷ is OR¹²; wherein when R⁶ is OR¹², R⁷ is H, R⁸ is C₁-C₁₅ alkyl; [symbol]
denotes an optional double bond linking the carbon adjacent to R⁷ with the carbon adjacent to R⁸;
R⁸ is H or C₁-C₁₅ alkyl having a straight or branched carbon chain, wherein the carbon chain optionally incorporates one or more double bonds, one or more triple bonds, one or more oxygen atoms and/or a terminal or non-terminal optionally substituted aryl group;
R¹¹ is lower alkyl, lower alkenyl or aralkyl;
R¹² is C₆-C₃₀ acyl having a straight or branched carbon chain optionally substituted with one or more hydroxy groups at positions 2 and/or 3 of the acyl group and/or an optionally substituted chain terminating aryl group and which optionally incorporates one or more double bonds, one or more triple bonds, and/or one or more optionally substituted arylene groups and wherein the carbon chain is optionally substituted with one or more deuterium atoms; wherein the optional substituents on the aryl and arylene groups may be selected from halogen, cyano, dialkylamino, C₁-C₆ amide, nitro, C₁-C₆ alkoxy, C₁-C₆ acyloxy and C₁-C₆ thioalkyl.

36. The combination according to claim 35 wherein A is selected from the group consisting of: 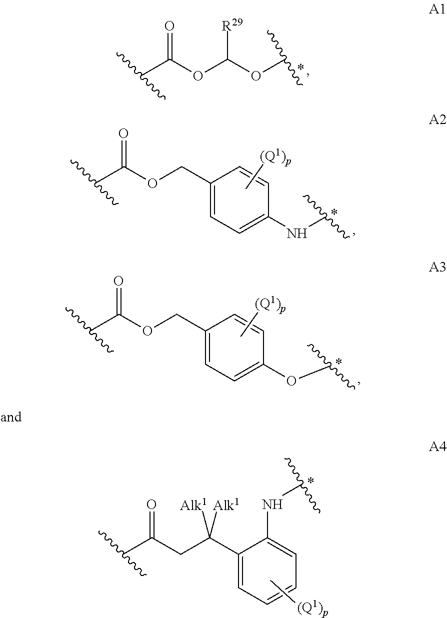
wherein * denotes a point of attachment of group A to group D;
each Q¹, the same or different, is independently selected from the group consisting of H, alkyl, alkoxy, halogen, nitro, aryl; or, together with the ring to which it is attached, forms a fused bicyclic aryl group;
p is an integer from 1 to 4;
Alk¹ is C₁-C₄ straight chain alkyl; and
R²⁹ is H or lower alkyl;
provided that A is A1 only when D is D1 and provided that A is A2 only when D is D2, D3 or D5 and provided that A is A3 only when D is D1, D3 or D4 and provided that A is A4 only when D is D2, D3 or D5.

37. The combination according to claim 35 wherein R¹⁵ is the side chain of L-citrulline or L-alanine.

38. The combination according to claim 36, wherein R¹⁶ is a side chain of one of an amino acid selected from: L-phenylalanine, L-valine, L-leucine, L-isoleucine, L-norleucine, L-methionine, L-tryptophan or L-tyrosine.

39. The combination according to claim 36 wherein D is D2 and R³² is (C₆-C₈)-alkylene.

40. The combination according to claim 35 wherein E is selected from the group consisting of E3, E4, E93, E94 and E97.

41. The combination according to claim 35 wherein G is selected from the group consisting of FFRK, GFLG and FKRL.

42. The combination according to claim 35 wherein R⁴ is CH₂OH.

43. The combination according to claim 35 wherein R⁶ is OH.

44. The combination according to claim 35 wherein R⁷ is OR¹².

45. The combination according to claim 44 wherein R¹² is C₆-C₃₀ acyl having a straight carbon chain containing no double bonds, triple bonds, oxygen atoms, aryl groups and which is unsubstituted or substituted with a terminating aryl group.

46. The combination according to claim 45 wherein R¹² is C₁₈-C₂₆ acyl having a straight carbon chain containing no double bonds, triple bonds, oxygen atoms, aryl groups and which is unsubstituted.

47. The combination according to claim 35 wherein R⁸ is C₁-C₁₅ alkyl.

48. The combination according to claim 47 wherein R⁸ is C₁₀ to C₁₄ alkyl having a straight or branched carbon chain containing no double bonds, triple bonds, oxygen atoms or aryl groups.

49. The combination according to claim 35 wherein J is a peptide antigen from a tumour, virus or bacteria.

50. The combination according to claim 35 wherein the TLR-9 agonist is a CpG oligodeoxynucleotide.

51. The combination according to claim 50 wherein the TLR-9 agonist is an oligodeoxynucleotide comprising a core sequence comprising a hexamer with a central unmethylated cytosine-phosphate-guanine (CpG) moiety, having a general formula RRCGYY; wherein R represents a purine; C represents a cytosine; G represents a guanine; and Y a pyrimidine.

52. The combination according to claim 51 wherein the TLR-9 agonist is an unmethylated single-stranded CpG oligodeoxynucleotide of about 15 to about 30 bases.

53. A pharmaceutical composition comprising a combination according to claim 35 and a pharmaceutically acceptable carrier or excipient.

54. A method of treating cancer in a subject, the method comprising administering to the subject a therapeutically effective amount of the combination of claim 35, wherein the combination is administered by intratumoural or peritumoural injection to a cancerous tumour.
